(19) United States
(12) Patent Application Publication
(10) Pub. No.: US 2026/0198503 A1
(43) Pub. Date: Jul. 16, 2026

(54) FUNGAL COMPOSITIONS AND METHODS OF USE THEREOF

(71) Applicant: Loam Bio Pty Ltd, Orange (AU)

(72) Inventors: Venkatachalam LAKSHMANAN, St. Paul, MN (US); James PRESNAIL, St. Paul, MN (US); Ray RILEY, St. Paul, MN (US); Tachir ORMESMITH, Orange (AU); Ahsanul HAQUE, Orange (AU); Tegan NOCK, Orange (AU)

(21) Appl. No.: 19/136,854

(22) PCT Filed: Dec. 5, 2023
(86) PCT No.: PCT/US2023/082547

§ 371 Date: Jun. 6, 2025

Related U.S. Application Data
(60) Provisional application No. 63/386,193, filed on Dec. 6, 2022.

(51) Int. Cl. A01N 63/38; A01N 63/30; A01P 21/00; C12N 1/145; C12R 1/645; C12R 1/885

(52) U.S. Cl. CPC A01N 63/38; A01N 63/30; A01P 21/00; C12N 1/145; C12R 2001/645; C12R 2001/885

(57) ABSTRACT

A method of increasing organic carbon in a soil is disclosed. The method includes inoculating the soil and/or a plant growing in the soil with one or more fungal strains wherein the one or more fungal strains has a nuclear ribosomal internal transcribed spacer 2 (ITS2) sequence that is at least 90% identical to the nucleotide sequence of any one of SEQ ID NOs: 1-11, wherein the one or more fungal strains are in an amount effective to increase organic carbon in the soil compared to a non-inoculated control soil. Also disclosed is a method of enhancing plant growth, comprising applying to a plant, a plant part, or the locus surrounding the plant with one or more fungal strains in an amount effective to enhance the growth of the plant as compared to an untreated control plant.

### CROSS REFERENCE TO RELATED APPLICATIONS

This application claims the benefit of U.S. Provisional Patent Application No. 63/386,193, filed on Dec. 6, 2022, the contents of which are incorporated herein by reference in their entirety.

### STATEMENT REGARDING ELECTRONIC FILING OF A SEQUENCE LISTING

A Sequence Listing in XML format, submitted under 37 C.F.R. § 1.821, entitled LOAM-F003-01WO.xml, 15,579 bytes in size, generated on Dec. 5, 2023, and filed via EFS-Web, is provided in lieu of a paper copy. This Sequence Listing is incorporated by reference into the specification for its disclosures.

### TECHNICAL FIELD

The present disclosure relates to methods and compositions for increasing carbon content in soil, mitigating atmospheric carbon dioxide, and increasing plant yield.

### BACKGROUND

Carbon dioxide and methane absorb and retain heat in the atmosphere, and therefore both gases play a pivotal role in the greenhouse effect. As methane is much more short-lived than carbon dioxide, carbon dioxide is often considered to be more important than that of methane to the greenhouse effect.

The life cycle of carbon includes the removal of carbon dioxide from the atmosphere by plants through photosynthesis. During the process of photosynthesis, the carbon dioxide gets absorbed through stroma of leaves, and the carbon dioxide is further converted into sugars. Such sugars become nutrients for plants and microbes present in the soil. Carbon enters back into the atmosphere in the form of carbon dioxide by respiration and combustion. Hence, a balanced amount of release and absorption of the carbon dioxide is an essential step for balancing the ecosystem.

Human activities such as combustion of fuels, overpopulation, forest degradation, soil erosion, etc. have led to an increase in atmospheric carbon dioxide. Approaches for sequestering carbon dioxide from the atmosphere therefore present an important component of a strategy for reducing or controlling atmospheric carbon dioxide. However, for this to be successful, there must also be a reduction in the release of carbon dioxide from soil back into the atmosphere.

Decay of plants, animals, and microbes into the soil can lead to the build-up of soil organic carbon (SOC), an essential nutrient which promotes physical stability of the structure of the soil, soil aeration, water drainage and retention, thus reducing soil erosion and nutrient leaching. However, intensive cultivation has also led to a decline in SOC, eventually making the land unsuitable for commercial crop production. As such, the benefits associated with SOC can be seen as two-fold, namely, the sequestration of atmospheric carbon, provided the carbon is retained by the soil, and the overall improvement of the soil quality.

It would be advantageous to develop compositions, treatments, and methods for increasing soil carbon in a manner that will produce more stable carbon in the soil by sequestering atmospheric carbon, as well as provide benefits to commercial crop plants.

### SUMMARY

The inventors have found that some species of fungi, and in particular endophytic fungi, are capable of both fixing carbon and increasing the portion of soil organic carbon stored in highly stable forms with increased soil carbon residence times while increasing the yield of crop plants, when the plant is physically associated with the fungus. In some aspects, the plant is inoculated with the fungus.

In some aspects, the present disclosure provides a method of increasing organic carbon in a soil, comprising: inoculating the soil and/or a plant growing in the soil with one or more fungal strains, wherein the one or more fungal strains has a nuclear ribosomal internal transcribed spacer 2 (ITS2) sequence that is at least 90% identical to the nucleotide sequence of any one of SEQ ID NOs: 1-11; and the one or more fungal strains are in an effective amount to increase organic carbon in the soil compared to a non-inoculated control soil.

In certain aspects, the present disclosure provides a method of increasing organic carbon with increased residence time in a soil, comprising: inoculating the soil and/or a plant growing in the soil with one or more fungal strains, wherein the one or more fungal strains has a nuclear ribosomal internal transcribed spacer 2 (ITS2) sequence that is at least 90% identical to the nucleotide sequence of any one of SEQ ID NOs: 1-11; and the one or more fungal strains are in an effective amount to increase organic carbon in the soil compared to a non-inoculated control soil.

In one aspect, the disclosed method further comprises an initial step of identifying the soil as having a soil organic carbon (SOC) level below a threshold. In some aspects, the threshold is a SOC level (% wt/wt) below 5%, 4%, 3%, 2%, 1%, 0.9%, 0.8%, 0.7%, 0.6%, 0.5%, 0.4%, 0.3%, 0.2%, or 0.1%. SOC levels (% wt/wt) are calculated with the following formula:

$$SOC ( \% wt / wt ) = ( \frac{Weight of Organic Carbon}{Weight of Soil Sample} ) \times 100$$

In certain aspects, the disclosure relates to a method for sequestering atmospheric carbon for storage as organic carbon in a soil, comprising: inoculating the soil and/or a plant growing in the soil with one or more fungal strains, wherein the one or more fungal strains has a nuclear ribosomal internal transcribed spacer 2 (ITS2) sequence that is at least 90% identical to the nucleotide sequence of any one of SEQ ID NOs: 1-11; and the one or more fungal strains are in an effective amount to increase sequestered atmospheric carbon in the soil compared to a non-inoculated control soil.

In other aspects, the disclosure provides a method of enhancing plant growth, comprising: applying to a plant, a plant part, or the locus surrounding the plant, one or more fungal strains, wherein the one or more fungal strains has a nuclear ribosomal internal transcribed spacer 2 (ITS2) sequence that is at least 90% identical to the nucleotide sequence of any one of SEQ ID NOs: 1-11; and the one or more fungal strains are in an effective amount to enhance the growth of the plant as compared to an untreated control plant. The term “enhance” as used herein means to increase or improve a property, characteristic, or value. In some aspects, plant growth is enhanced by increased phosphate solubilization or siderophore production by the one or more fungal strains.

In one aspect, the plant exhibits at least one of increased root number, increased root length, increased root mass, increased root volume, increased leaf area, increased leaf number, increased pod number, increased plant height, increased shoot mass, increased chlorophyll content, increased nodulation, and increased yield, as compared to an untreated control plant.

In certain aspects, the one or more fungal strains utilized in the methods of the present disclosure are selected from the group consisting of: Periconia macrospinosa AU-7083 (NMI Accession No. V22/019796), Acrocalymma vagum US-738 (ATCC Accession No. PTA-127449), Acrocalymma vagum US-445 (ATCC Accession No. PTA-127444), Leptodontidium orchidola US-210 (ATCC Accession No. PTA-127441), Leptodontidium orchidola US-70 (ATCC Accession No. PTA-127439), Leptodontidium orchidola US-1188 (ATCC Accession No. PTA-127640), Leptodontidium orchidola US-1202 (ATCC Accession No. PTA-127639), Clonostachys rosea US-114 (ATCC Accession No. PTA-127443), Clonostachys rosea US-712 (ATCC Accession No PTA-127446), Trichoderma hamatum US-724 (ATCC Accession No. PTA-127448), Trichoderma longipile/spirale US-77 (ATCC Accession No. PTA-127440), and a mutant thereof having all identifying characteristics of the respective strain.

In other aspects, the soil and/or plant are non-native to the one or more fungal strains, In some embodiments, the non-native plant is either a monocotyledonous plant (aka a monocot) or a dicotyledonous plant (aka a dicot). In one aspect, the non-native plant is selected from the group consisting of wheat, rice, corn (maize), canola, rye, oats, barley, sorghum, millet, flax, hemp, jute, cotton, soybeans, alfalfa, clover, peanuts, lentils, lupins, peas, and chickpea. In another aspect, the non-native plant is selected from the group consisting of Lucerne, arrow leaf clover, Balansa clover, chicory, plantain, Phalaris, cocksfoot, fescue, prairie grass, Warrego summer grass, Italian rye grass, perennial rye grass, Biserrula, Serradella, Gland clover, Bladder clover, switchgrass, radish, medic, buckwheat, cow pea, lablab, sunn hemp, sunflower, tillage radish, and subterranean clover.

In yet other aspects, the organic carbon in the soil is increased in the stable forms of Aggregate Carbon fraction (AggC), aggregate occluded Particulate Organic Carbon (oPOC), and/or Mineral-Associated Organic Carbon (MAOC).

In certain aspects, the disclosed method further comprises applying the fungal strain at a rate of between 10 CFU/seed and 10,000 CFU/seed, between 50 CFU/seed and 10,000 CFU/seed, between 100 CFU/seed and 10,000 CFU/seed, between 200 CFU/seed and 10,000 CFU/seed, between 300 CFU/seed and 10,000 CFU/seed, between 400 CFU/seed and 10,000 CFU/seed, between 500 CFU/seed and 10,000 CFU/seed, between 600 CFU/seed and 10.000 CFU/seed, between 700 CFU/seed and 10,000 CFU/seed, between 800 CFU/seed and 10,000 CFU/seed, between 900 CFU/seed and 10,000 CFU/seed, between 1,000 CFU/seed and 10,000 CFU/seed, between 1,500 CFU/seed and 10,000 CFU/seed, between 2,000 CFU/seed and 10,000 CFU/seed, between 2,500 CFU/seed and 10,000 CFU/seed, between 3,000 CFU/seed and 10,000 CFU/seed, between 3,500 CFU/seed and 10,000 CFU/seed, between 4,000 CFU/seed and 10,000 CFU/seed, between 4,500 CRU/seed and 10,000 CFU/seed, or between 5,000 CFU/seed and 10,000 CFU/seed.

In other aspects, the disclosed method further comprises applying the fungal strain at a rate of between 1 g/Ha and 1,000 g/Ha, between 10 g/Ha and 1,000 g/Ha, between 20 g/Ha and 1,000 g/Ha, between 30 g/Ha and 1,000 g/Ha, between 40 g/Ha and 1,000 g/Ha, between 50 g/Ha and 1,000 g/Ha, between 60 g/Ha and 1,000 g/Ha, between 70 g/Ha and 1,000 g/Ha, between 80 g/Ha and 1,000 g/Ha, between 90 g/1 Ha and 1,000 g/Ha, between 100 g/Ha and 1,000 g/Ha, between 1 g/Ha and 500 g/Ha, between 10 g/Ha and 500 g/Ha, between 20 g/Ha and 500 g/Ha, between 30 g/Ha and 500 g/Ha, between 40 g/Ha and 500 g/1a, between 50 g/Ha and 500 g/Ha, between 60 g/Ha and 500 g/Ha, between 70 g/Ha and 500 g/Ha, between 80 g/Ha and 500 g/Ha, between 90 g/Ha and 500 g/Ha, or between 100 g/Ha and 500 g/Ha.

In other aspects, the present disclosure provides a plant, plant part or plant seed associated with a composition comprising: one or more fungal strains having a nuclear ribosomal internal transcribed spacer 2 (ITS2) sequence that is at least 90% identical to the nucleotide sequence of any one of SEQ ID NOs: 1-11; and an agriculturally acceptable carrier; wherein the composition is applied or coated on at least a portion of an outer surface of the plant, plant part or plant seed.

In one aspect, the one or more fungal strains utilized in the compositions of the present disclosure are selected from the group consisting of: Periconia macrospinosa AU-7083 (NMI Accession No. V22/019796), Acrocalymma vagum US-738 (ATCC Accession No. PTA-127449), Acrocalymma vagum US-445 (ATCC Accession No. PTA-127444), Leptodontidium orchidola US-210 (ATCC Accession No. PTA-127441), Leptodontidium orchidola US-70 (ATCC Accession No. PTA-127439), Leptodontidium orchidola US-1188 (ATCC Accession No. PTA-127640), Leptodontidium orchidola US-1202 (ATCC Accession No. PTA-127639), Clonostachys rosea US-114 (ATCC Accession No. PTA-127443), Clonostachys rosea US-712 (ATCC Accession No PTA-127446), Trichoderma hamatum US-724 (ATCC Accession No. PTA-127448), Trichoderma longipile/spirale US-77 (ATCC Accession No. PTA-127440), and a mutant thereof having all identifying characteristics of the respective strain.

In certain aspects, the plant, plant part or plant seed is non-native to the one or more fungal strains. In one aspect, the non-native plant, plant part or plant seed is selected from the group consisting of wheat, rice, corn (maize), canola, rye, oats, barley, sorghum, millet, flax, hemp, jute, cotton, soybeans, alfalfa, clover, peanuts, lentils, lupins, peas, and chickpea, in another aspect, the non-native plant, plant part or plant seed is a pasture crop or cover crop selected from the group consisting of Lucerne, arrow leaf clover, Balansa clover, chicory, plantain, Phalaris, cocksfoot, fescue, prairie grass, Warrego summer grass, Italian rye grass, perennial rye grass, Biserrula, Serradella, Gland clover, Bladder clover, switchgrass, radish, medic, buckwheat, cow pea, lablab, sunn hemp, sunflower, tillage radish, and subterranean clover.

In yet other aspects, (a) the composition is formulated as a solid, liquid or gel; (b) the composition is formulated as a powder, pellet or granules; or (c) the composition is formulated as an emulsion, colloid, suspension or solution. In some aspects, the composition is formulated as a powder, a pellet, granules, emulsion, colloid, suspension or solution.

In certain aspects, the one or more fungal strains are present in the composition at a concentration of at least 10³ CFU per milliliter or gram.

In yet other aspects, the one or more fungal strains are present in the composition at a concentration of at least 10¹ CFU per millilitre or gram, at least 10² CFU pet millilitre or gram, at least 10³ CFU per millilitre or gram, at least 10⁴ CFU per millilitre or gram, at least 10⁴ CFU per millilitre or gram, or at least 10′ CFU per millilitre or gram. In one aspect, the one or more fungal strains are present in the composition at a concentration of at least 10³ CFU per millilitre or gram.

In some aspects, the one or more fungal strains are present in the composition at a concentration of 10² to 10¹² CFU/g, 10³ to 10¹² CFU/g, 10⁴ to 10¹² CFU/g, 10⁵ to 10¹² CFU/g, 10⁶ to 10¹² CFU/g, 10⁷ to 10¹² CFU/g, 10⁹ to 10¹² CFU/g, or 10⁹ to ₁₀¹² CFU/g.

In one aspect, the present disclosure provides a bag or container comprising plant seed described herein, In another aspect, the present disclosure provides a kit comprising plant seed described herein.

In yet another aspect, the present disclosure relates to a cell or a biologically pure culture of one or more fungal strains selected from the group consisting of Periconia macrospinosa AU-7083 (NMI Accession No. V22/019796), Acrocalymma vagum US-738 (ATCC Accession No. PTA-127449), Acrocalymma vagum US-445 (ATCC Accession No. PTA-127444), Leptodontidium orchidola US-210 (ATCC Accession No. PTA-127441), Leptodontidium orchidola US-70 (ATCC Accession No. PTA-127439), Leptodontidium orchidola US-1188 (ATCC Accession No. PTA-127640), Leptodontidium orchidola US-1202 (ATCC Accession No. PTA-127639), Clonostachys rosea US-114 (ATCC Accession No. PTA-127443), Clonostachys rosea US-712 (ATCC Accession No PTA-127446), Trichoderma hamatum US-724 (ATCC Accession No. PTA-127448), Trichoderma longipile/spirale US-77 (ATCC Accession No. PTA-127440), and a mutant thereof having all identifying characteristics of the respective strain.

In one aspect, the present disclosure provides an agricultural composition comprising a cell or a biologically pure culture described herein and, optionally, an agriculturally acceptable carrier. In some aspects, the agricultural composition is applied or coated on at least a portion of an outer surface of a plant, plant part or plant seed.

In other aspects, the present disclosure relates to a bioorganic soil conditioner comprising a cell or a biologically pure culture described herein and, optionally, an agriculturally acceptable carrier.

### BRIEF DESCRIPTION OF THE DRAWINGS

FIG. 1 depicts representative images of fungal colonies cultured on solid Pikovskaya (PVK) media containing insoluble superphosphate with clear zones of dissolved phosphate indicating phosphate solubilization by the fungal cells.

FIG. 2 depicts siderophore production by Leptodontidium orchidicola US-70 (ATCC Accession No. PTA-127439) indicated by the presence of a ring around the edge of the fungal colony on a chrome azurol S (CAS) agar plate.

FIG. 3 depicts increases in soil carbon after 8 weeks of growth of Bennett wheat seeds treated with Periconia macrospinosa AU-7083 (NMI Accession No. V22/019796) or Periconia macrospinosa DMTR-CTR-1852 (NMI Accession No. V22/006358),

FIG. 4 depicts increases in soil carbon after 8 weeks of growth of Condo wheat seeds treated with Periconia macrospinosa AU-7083 (NMI Accession No. V22/019796) or Periconia macrospinosa DMTR-CTR-1852 (NMI Accession No. V22/006358).

### DETAILED DESCRIPTION

The present disclosure relates to methods and related technologies for increasing soil organic carbon in a soil and/or increasing yield of a crop plant. The method comprises inoculating the soil and/or the plant with an effective amount of one or more compatible, non-pathogenic strains of fungal species.

It will be appreciated that the strains of fungi will be fungal strains that are crop-compatible with the crop plant to which they are to be applied, but the crop need not necessarily be a native host of the fungi. A fungal strain that is crop-compatible with a crop plant is a strain that is non-pathogenic to that crop plant. Methods for assessing whether a strain of fungus is non-pathogenic to a particular crop plant is known in the art.

An increase in soil organic carbon is an increase in the amount of organic carbon in soil associated with the crop plant inoculated with the one or more fungal species relative to the amount of organic carbon in uninoculated soil. In this context, the soil associated with the crop plant is soil surrounding the roots of the crop plant and from which the crop plant derives nutrients. An increase in plant yield is an increase in fruit, grain or vegetative tissue production of the plant relative to that of a plant that has not been treated with the one or more fungal species described herein. For example, an increase in yield of a soybean plant is an increase in the number and/or weight of seed pods produced by a soybean plant relative to that of an untreated soybean plant.

The inventors have found that growing a crop plant that has been inoculated with certain crop-compatible fungal strains results in an increase in soil organic carbon and/or an increase in yield of crop plants.

The inventors have found that various crop plants inoculated with fungal species, and in particular, endophytic fungal species, exhibit increased yield relative to uninoculated plants. The inventors have further found that the soil in which these plants are grown has increased organic carbon content relative to soil in which uninoculated plants are grown.

The term “endophytic” relates to a microbe that generally lives within a plant for at least part of its lifecycle, often due to the microbe being able to grow inward into plant tissues in finger-like projections from a superficial site of origin. These fungi can infiltrate plant living tissues for at least a portion of the fungal life cycle often without causing any apparent diseases or harm to the plant that is a native host, in that they are generally not pathogenic to their native hosts. It would be understood that the one or more fungal genus, species or strains of the methods described herein can exist during some portion of the fungal life cycle within the roots of a plant host as an endophyte and in other parts of its life cycle within the soil and will typically alternate or cycle between a root endophytic phase and a free-living soil phase.

Though some endophytic fungi are known for enriching the organic carbon in the soil each fungal species will generally behave differently when associated with different, and/or non-native plant hosts and/or soil environments and will stabilize the organic carbon with different efficiency.

In some embodiments, the one or more fungal species has a nuclear ribosomal internal transcribed spacer 2(ITS2) sequence that is at least 90% identical, at least 91%, least 92%, at least 93%, at least 94%, at least 95%, at least 96%, at least 97%, at least 98%, or at least 99% identical, or 100%4 identical, with the nucleotide sequence of SEQ ID No: 1, 2, 3, 4, 5, 6, 7, 8, 9, 10, or It.

The terms “identical” or “% identical,” in the context of two or more nucleic acids refers to two or more sequences that are the same or have a specified percentage of nucleotides that are the same (i.e., 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCB1 web site http://www.ncbi.nlm.nih.gov/BLAST/, or the like).

Algorithms for determining % identity are known in the art. An example of an algorithm that is suitable for determining percent sequence identity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information.

A “soil conditioner” is to be understood to denote a mixture of substances or a blend that can be added directly to soil to improve soil characteristics or can be added to an agricultural or fertilizer composition, which, in turn, is added to a soil. The soil conditioner can be applied to any type of soil, including black cotton soil, saline soil, medium to high saline soil, yellow soil, sandy soil, loamy soil, alluvial soil (delta soil), lava soil, topsoil, and subsoil that can be used in crop/plant production.

As used herein, the term “effective amount” means a sufficient quantity of a substance (e.g. fungus) to promote an increase in soil carbon and/or yield of a plant. This term is not to be construed to limit the disclosure to a specific quantity, e.g., number of fungal cells; rather the present disclosure encompasses any amount of the one or more fungal species that is sufficient to achieve the stated purpose. The amount of the one or more fungal species should not be so large as to cause adverse effects in the plant. Generally, the amount of the one or more fungal species may be varied with the way in which the fungi are applied (e.g., to the soil, to the seed or to the seedling) and can be determined by a person skilled in the art.

Throughout the specification and claims, unless the context requires otherwise, the term “substantially” or “about” will be understood to not be limited to the value for the range qualified by the terms. For example, the term “about” may include a range that is ±5%, ±2.5% or ±1% of the value to which the term is applied.

### Deposited Fungal Strains

Biological deposits of each of the fungal strains listed in Table 1 were made on the dates shown at the American Type Culture Collection (ATCC®), located at 10801 University Blvd., Manassas, VA 20110, USA, or the National Measurement Institute (NMI), 1/153 Bertie Street, Port Melbourne, Victoria 3207, Australia, under the provisions of the Budapest Treaty and assigned by each International Depositary Authority (IDA) the accession numbers indicated, Upon issuance of a patent, all restrictions upon the deposits will be irrevocably removed. The deposits are intended to meet the requirements of 37 CPR. §§ 1.801-1.809. The deposits will be maintained in the IDAs for a period of 30 years, or 5 years after the last request, or for the effective, enforceable life of the patent, whichever is longer, and will be replaced, if necessary, during that period, and the requirements of 37 CFR §§ 1.801-1.809 are met.

TABLE 1

| Strain Number | Species | IDA | Date of Deposit | Accession No. |
|---|---|---|---|---|
| AU-7083 | Periconia | NMI | 21 Oct. 2022 | V22/019796 |
| | macrospinosa | | | |
| US-738 | Acrocalymma | ATCC | 23 Nov. 2022 | PTA-127449 |
| | vagum | | | |
| US-445 | Acrocalymma | ATCC | 23 Nov. 2022 | PTA-127444 |
| | vagum | | | |
| US-210 | Leptodontidium | ATCC | 23 Nov. 2022 | PTA-127441 |
| | orchidicola | | | |
| US-70 | Leptodontidium | ATCC | 23 Nov. 2022 | PTA-127439 |
| | orchidicola | | | |
| US-1188 | Leptodontidium | ATCC | Sep. 7, 2023 | PTA-127640 |
| | orchidicola | | | |
| US-1202 | Leptodontidium | ATCC | Sep. 7, 2023 | PTA-127639 |
| | orchidicola | | | |
| US-114 | Clonostachys | ATCC | 23 Nov. 2022 | PTA-127443 |
| | rosea | | | |
| US-712 | Clonostachys | ATCC | 23 Nov. 2022 | PTA-127446 |
| | rosea | | | |
| US-724 | Trichoderma | ATCC | 23 Nov. 2022 | PTA-127448 |
| | hamatum | | | |
| US-77 | Trichoderma | ATCC | 23 Nov. 2022 | PTA-127440 |
| | longipile | | | |
| | spirale | | | |

It would be understood that fungal strains from the same species as those described herein would have similar desirable attributes and are encompassed by the treatments and methods of the present invention.

### Internal Transcribed Spacer 2 (ITS2) Sequences of Fungal Strains

In one aspect, the fungal strain comprises a nuclear ribosomal internal transcribed spacer 2 (ITS2) sequence that is at least 80% identical to the nucleotide sequence of any one of SEQ ID NOs: 1-11 (see Table 2).

In one aspect, the fungal strain belongs to the genus Periconia. In another aspect, the fungal strain comprises a nuclear ribosomal internal transcribed spacer 2 (ITS2) sequence that is at least 95% identical, at least 96% identical, at least 97% identical, at least 98%/0 identical, or at least 99% identical to the nucleotide sequence of SEQ ID NO: 1.

In one aspect, the fungal strain belongs to the genus Acrocalymma. In another aspect, the fungal strain comprises a nuclear ribosomal internal transcribed spacer 2 (ITS2) sequence that is at least 95% identical, at least 96% identical, at least 97% identical, at least 98% identical, or at least 99% identical to the nucleotide sequence of SEQ ID NO: 2 or 3.

In one aspect, the fungal strain belongs to the genus Leptodontidium, In another aspect, the fungal strain comprises a nuclear ribosomal internal transcribed spacer 2 (ITS2) sequence that is at least 95% identical, at least 96% identical, at least 97% identical, at least 98% identical, or at least 99% identical to the nucleotide sequence of SEQ ID NO: 4 or 5.

In one aspect, the fungal strain belongs to the genus Clonostachys. In another aspect, the fungal strain comprises a nuclear ribosomal internal transcribed spacer 2 (ITS2) sequence that is at least 95% identical, at least 96% identical, at least 97% identical, at least 98% identical, or at least 99% identical to the nucleotide sequence of SEQ ID NO: 6 or 7.

In one aspect, the fungal strain belongs to the genus Trichoderma. In another aspect, the fungal strain comprises a nuclear ribosomal internal transcribed spacer 2 (ITS2) sequence that is at least 95% identical, at least 96% identical, at least 97% identical, at least 98% identical, or at least 99% identical to the nucleotide sequence of SEQ ID NO: 8 or 9.

TABLE 2

| SEQ ID | Strain | | |
|---|---|---|---|
| No. | Number | Species | ITS2 sequence |
| 1 | AU-7083 | Periconia | GCATCGATGAAGAACGCAGCGAAATGC |
| | | macrospinosa | GATAAGTAGTGTGAATTGCAGAATTCA |
| | | | GTGAATCATCGAATCTTTGAACGCACAT |
| | | | TGCGGCCATAGGTATTCCTTTGGCCATG |
| | | | CCTGTTCGAGCGTCATTTACACCCTCAA |
| | | | GCCTAGCTTGGTGTTGGGCGTCTGTCCC |
| | | | GCCGTTTTCGCGCGCGGACTCGCCTCAA |
| | | | AGTCATTGGCGGCGGTCGTGCCGGCCCC |
| | | | CTCGCGCAGCACATTTGCGCTTCTCGGA |
| | | | GGCCCGGCGGATCCGCGCTCCAGCAAG |
| | | | ACCTTTCACGACTTGACCTCGGATCAGG |
| | | | TAGGGATACCCGCTGAACTTAAGCATAT |
| | | | CAATAAGCGGAGGA |
| 2 | US-738 | Acrocalymma | TGCGGGAACACCCTAAAGACCTCAACA |
| | | vagum | CCAAGCGTCATGGGAAACCATGGCGTG |
| | | | GCCGAGCTAATAGCCCTGGGTATGGTA |
| | | | ACAGCTTGAGGTATGAAGCCTTCGCAA |
| | | | GGAGGCCGAAATGGGCAATCCGCAGCC |
| | | | AAGTCCTAACGTGCTCGAAACCGAGTG |
| | | | CCATGGATGCTGTTCACAGGCCAAATGG |
| | | | TAGTGGGTGACTCTTGCGAGTTGCTTAA |
| | | | GATATGGTCGGGCCCCTTCAGAAATGTG |
| | | | GGGGATAAGCTTACGCTTCTCCAAACCG |
| | | | TTCCGTAGGTGAACCTGCGGAAGGATC |
| | | | ATTAACGATTTCGGTGTAAAAAACCGTT |
| | | | TTCTACCTATGTCTACGCGTACCACATG |
| | | | TTTCCTCGGGGGGCTTGCCCCCCGCTAG |
| | | | GACCCTTTATCAAACCTTTTTGTAATAG |
| | | | CAGTCAGCGTCTGATACTAAGTTAATTA |
| | | | TTAAAACTTTCAACAATGGATCTCTTGG |
| | | | TTCTGGCATCGATGAAGAACGCAGCGA |
| | | | AATGCGATAAGTAGTGTGAATTGCAGA |
| | | | ATTCAGTGAATCATCGAATCTTTGAACG |
| | | | CACATTGCGCCCCTTGGTATTCCATGGG |
| | | | GCATGCCTGTTCGAGCGTCATTTGAACC |
| | | | CTCAAGCTCTGCTTGGTGTTGGGTGTTT |
| | | | GTCCCGCCATTGCGCGTGACTCGCCTTA |
| | | | AGCAATTGGCAGCCATGTAATCCCGCCT |
| | | | TTGAGCGCAGCACATTGCGTACTCTCTA |
| | | | CTGGACATGGGCATCCAGAAGCCTTATT |
| | | | TTT |
| 3 | US-445 | Acrocalymma | TCCTCCGCTTATTGATATGCTTAAGTTC |
| | | vagum | AGCGGGTATCCCTACCTGATCCGAGGTC |
| | | | AAGAGTAAAAAATAAGGCTTCTGGATG |
| | | | CCCATGTCCCAGTAGAGAGTACGCAAT |
| | | | GTGCTGCGCTCAAAGCCGGATTACATGG |
| | | | CTGCCAATTGCTTTAAGGCGAGTCCACG |
| | | | CGCAATGGCGGGACAAACACCCAACAC |
| | | | CAAGCAGAGCTTGAGGGTTCAAATGAC |
| | | | GCTCGAACAGGCATGCCCCATGGAATA |
| | | | CCAAGGGGCGCAATGTGCGTTCAAAGA |
| | | | TTCGATGATTCACTGAATTCTGCAATTC |
| | | | ACACTACTTATCGCATTTCGCTGCGTTC |
| | | | TTCATCGATG |
| 4 | US-210 | Leptodontidium | TCCTCCGCTTATTGATATGCTTAAGTTC |
| | | orchidicola | AGCGGGTATCCCTACCTGATCCGAGGTC |
| | | | AACCTTAAAAAAATTAGGGGTTGCTGG |
| | | | CAAGTAGACCTACCGGACTCAATCGCG |
| | | | AGGAGTATTACTACGCGTAGAGCCGAC |
| | | | AGGCACCGCCACTGATTTTAGGGGCCGC |
| | | | GAAACCGCGAACCCCAATACCAAGCGA |
| | | | GAGCTTGAGTGGTTATAATGACGCTCGA |
| | | | ACAGGCATGCCCCCCGGAATACCAGAG |
| | | | GGCGCAATGTGCGTTCAAAGATTCGATG |
| | | | ATTCACTGAATTCTGCAATTCACATTAC |
| | | | TTATCGCATTTCGCTGCGTTCTTCATCGA |
| | | | TG |
| 5 | US-70 | Leptodontidium | GCATCGATGAAGAACGCAGCGAAATGC |
| | | orchidicola | GATAAGTAATGTGAATTGCAGAATTCA |
| | | | GTGAATCATCGAATCTTTGAACGCACAT |
| | | | TGCGCCCTCTGGTATTCCGGGGGGCATG |
| | | | CCTGTTCGAGCGTCATTATAACCACTCA |
| | | | AGCTCTCGCTTGGTATTGGGGTTCGCGG |
| | | | TTTCGCGGCCCCTAAAATCAGTGGCGGT |
| | | | GCCTGTCGGCTCTACGCGTAGTAATACT |
| | | | CCTCGCGATTGAGTCCGGTAGGTCTACT |
| | | | TGCCAGCAACCCCTAATTTTTTTTTAAG |
| | | | GTTGACCTCGGATCAGGTAGGGATACCC |
| | | | GCTGAACTTAAGCATATCAATAAGCGG |
| | | | AGG |
| 6 | US-1188 | Leptodontidium | CTGCGGAGGATCATTACTAGAGCAAAG |
| | | orchidicola | GATAGACAGCCCCCGCGGAGCTCGCTC |
| | | | CCGGGGCCACCCTACTCCGGTAGGGTTT |
| | | | AGAGTCGTCGGGCCTCTCGGAGAAGCT |
| | | | CGGTCCTGAACTCCACCCTTGAATAAAC |
| | | | TACCTTTGTTGCTTTGGCGGGCCGCCTC |
| | | | GTGCCAGCGGCTTCGGCTGTTGAGTGCC |
| | | | CGCCAGAGGACCACAACTCTTGTTTTTA |
| | | | GTGATGTCTGAGTACTATATAATAGTTA |
| | | | AAACTTTCAACAACGGATCTCTTGGTTC |
| | | | TGGCATCGATGAAGAACGCAGCGAAAT |
| | | | GCGATAAGTAATGTGAATTGCAGAATTC |
| | | | AGTGAATCATCGAATCTTTGAACGCACA |
| | | | TTGCGCCCTCTGGTATTCCGGGGGGCAT |
| | | | GCCTGTTCGAGCGTCATTATAACCACTC |
| | | | AAGCTCTCGCTTGGTATTGGGGTTCGCG |
| | | | GTTTCGGGGCTCCTAAAATCAGTGGCGG |
| | | | TGCCTATCGGCTCTACGCGTAGTAATAC |
| | | | TCCTCGCGATTGAGTCCGGTAGGTCTAC |
| | | | TTGCCAGCAACCCCTAATTTTTTAAGGT |
| | | | TGACCTCGGATCAGGTAGGGATACCCG |
| | | | CTGAACTTAAGCATATCA |
| 7 | US-1202 | Leptodontidium | CTTGGTCATTTAGAGGAAGTAAAAGTCG |
| | | orchidicola | TAACAAGGTTTCCGTAGGTGAACCTGCG |
| | | | GAAGGATCATTACTAGAGCAAAGGATA |
| | | | GACAGCGCCCGCGGAGCTCGCTCCCGG |
| | | | GGCTACCCTACTCCGGTAGGGTTTAGAG |
| | | | TCGTCGGGCCTCTCAGAGAAGCTCGGTC |
| | | | CTGAACTCCACCCTTGAATAAACTACCT |
| | | | TTGTTGCTTTGGCGGGCCGCCTCGTGCC |
| | | | AGCGGCTTTGGCTGTTGAGTGCCCGCCA |
| | | | GAGGACCACAACTCTTGTTTTTAGTGAT |
| | | | GTCTGAGTACTATATAATAGTTAAAACT |
| | | | TTCAACAACGGATCTCTTGGTTCTGGCA |
| | | | TCGATGAAGAACGCAGCGAAATGCGAT |
| | | | AAGTAATGTGAATTGCAGAATTCAGTG |
| | | | AATCATCGAATCTTTGAACGCACATTGC |
| | | | GCCCTCTGGTATTCCGGGGGGCATGCCT |
| | | | GTTCGAGCGTCATTATAACCACTCAAGC |
| | | | TCTCGCTTGGTATTGGGGTTCGCGGTTT |
| | | | CGCGGCCCCTAAATTCAGTGGCGGTGCC |
| | | | TATCGGCTCTACGCGTAGTAATACTCCT |
| | | | CGCGATTGAGTCCGGTAGGTCTACTTGC |
| | | | CAGTAACCCCTAATTTTTTAAGGTTGAC |
| | | | CTCGGATCAGGTAGGGATACCCGCTGA |
| | | | ACTTAAGCATATCAATAAGCGGAGG |
| 8 | US-114 | Clonostachys | GCATCGATGAAGAACGCAGCGAAATGC |
| | | rosea | GATAAGTAATGTGAATTGCAGAATTCA |
| | | | GTGAATCATCGAATCTTTGAACGCACAT |
| | | | TGCGCCCGCCAGTATTCTGGCGGGCATG |
| | | | CCTGTCTGAGCGTCATTTCAACCCTCAT |
| | | | GCCCCTAGGGCGTGGTGTTGGGGATCG |
| | | | GCCAAAGCCCGCGAGGGACGGCCGGCC |
| | | | CCTAAATCTAGTGGCGGACCCGTCGTGG |
| | | | CCTCCTCTGCGAAGTAGTGATATTCCGC |
| | | | ATCGGAGAGCGACGAGCCCCTGCCGTT |
| | | | AAACCCCCAACTTTCCAAGGTTGACCTC |
| | | | AGATCAGGTAGGAATACCCGCTGAACT |
| | | | TAAGCATATCAATAAGCGGAGG |
| 9 | US-712 | Clonostachys | GCGGAGGGATCATTACCGAGTTTACAA |
| | | rosea | CTCCCAAACCCATGTGAACATACCTACT |
| | | | GTTGCTTCGGGGGGATTGCCCCGGGCGC |
| | | | CTCGTGTGCCCCGGATCAGGCGCCCGCC |
| | | | TAGGAAACTTAATTCTTGTTTTATTTTGG |
| | | | AATCTTCTGAGTAGTTTTTACAAATAAA |
| | | | TAAAAACTTTCAACAACGGATCTCTTGG |
| | | | TTCTGGCATCGATGAAGAACGCAGCGA |
| | | | AATGCGATAAGTAATGTGAATTGCAGA |
| | | | ATTCAGTGAATCATCGAATCTTTGAACG |
| | | | CACATTGCGCCCGCCAGTATTCTGGGGG |
| | | | GCATGCCTGTCTGAGCGTCATTTCAACC |
| | | | CTCATGCCCCTAGGGCGTGGTGTTGGGG |
| | | | ATCGGCCAAAGCCCGCGAGGGACGGCC |
| | | | GGCCCCTAAATCTAGTGGGGGACCCGTG |
| | | | GTGGCCTCCTCTGCGAAGTAGTGATATT |
| | | | CCGCATCGGAGAGCGACGAGCCCCTGC |
| | | | CGTTAAACCCCCAACTTTCCAAGGTTGA |
| | | | CCTCAGATCAGGTAGGAATACCCGCTG |
| | | | AACTTAAGCATATCAA |
| 10 | US-724 | Trichoderma | CGGAGGGATCATTACCGAGTTTACAACT |
| | | hamatum | CCCAAACCCAATGTGAACGTTACCAAA |
| | | | CTGTTGCCTCGGGGGGGTCACGCCCCGG |
| | | | GTGCGTAAAAGCCCCGGAACCAGGCGC |
| | | | CCGCCGGAGGAACCAACCAAACTCTTTC |
| | | | TGTAGTCCCCTCGCGGACGTATTTCTTA |
| | | | CAGCTCTGAGCAAAAATTCAAAATGAA |
| | | | TCAAAACTTTCAACAACGGATCTCTTGG |
| | | | TTCTGGCATCGATGAAGAACGCAGCGA |
| | | | AATGCGATAAGTAATGTGAATTGCAGA |
| | | | ATTCAGTGAATCATCGAATCTTTGAACG |
| | | | CACATTGCGCCCGCCAGTATTCTGGCGG |
| | | | GCATGCCTGTCCGAGCGTCATTTCAACC |
| | | | CTCGAACCCCTCCGGGGGATCGGCGTTG |
| | | | GGGATCGGGACCCCTCACCGGGTGCCG |
| | | | GCCCTGAAATACAGTGGCGGTCTCGCCG |
| | | | CAGCCTCTCCTGCGCAGTAGTTTGCACA |
| | | | ACTCGCACCGGGAGCGCGGCGCGTCCA |
| | | | CGTCCGTAAAACACCCAACTTCTGAAAT |
| | | | GTTGACCTCGGATCAGGTAGGAATACCC |
| | | | GCTGAACTTAAGCATATCAATAAGGGG |
| | | | AGGA |
| 11 | US-77 | Trichoderma | GCATCGATGAAGAACGCAGCGAAATGC |
| | | longipile | GATAAGTAATGTGAATTGCAGAATTCA |
| | | spirale | GTGAATCATCGAATCTTTGAACGCACAT |
| | | | TGCGCCCGCCAGTATTCTGGCGGGCATG |
| | | | CCTGTCCGAGCGTCATTTCAACCCTCGA |
| | | | ACCCCTCCGGGGGGTCGGCGTTGGGGA |
| | | | TCGGCCCTTTACGGGGCCGGCCCCGAAA |
| | | | TACAGTGGCGGTCTCGCCGCAGCCTCTC |
| | | | CTGCGCAGTAGTTTGCACACTCGCATCG |
| | | | GGAGCGCGGCGCGTCCATTGCCGTTAA |
| | | | ACACCCAACTTTCTGAAATGTTGACCTC |
| | | | GGATCAGGTAGGAATACCCGCTGAACT |
| | | | TAAGCATATGAATAAGCGGAGG |

### Crop Plants

A “plant” means any plant of economic importance and includes cereals (such as wheat, barley, rye, triticale, millet, oats), maize (corn) canola, cotton, soya bean, rice, potatoes, sunflowers, beans, coffee, beets (e.g. sugar beets and fodder beets), peanuts, oilseed rape, poppies, olives, coconuts, cacao, sugar cane, tobacco, vegetables (such as tomatoes, cucumbers, onions and lettuce), lawn and ornamental plants. In a preferred embodiment, the plant is a crop plant. “Crop plant” generally means any cultivated plant that is grown to produce a harvested horticultural product that is grown for sale and/or profit, as well as subsistence crops which may be grown to support other agricultural products, such as livestock. The crop plant may be any crop plant of agronomic importance which is cultivated for food, animal feed, fiber, fuel, and/or industrial purposes. The crop plant may vary from region to region worldwide, wherein the variance may depend on factors such as dietary requirements and environmental conditions.

“Monocotyledon,” “monocotyledonous,” or “monocot” are understood to mean grass and grass-like flowering plants, the seeds of which typically contain only one embryonic leaf, or cotyledon.

“Dicotyledon,” or “dicotyledonous,” or “dicot” are understood to mean plants the seeds of which typically have two embryonic leaves, or cotyledons.

“Plant cultivars” are understood to mean plants which have new properties (“traits”) and have been obtained by conventional breeding, by mutagenesis or by recombinant DNA techniques, They can be cultivars, varieties, bio- or genotypes.

“Plant parts” are understood to mean all parts and organs of plants above and below the ground, such as shoots, leaves, needles, stalks, stems, flowers, fruit bodies, fruits, seeds, roots, tubers and rhizomes. The plant parts also include harvested material and vegetative and generative propagation material, for example cuttings, tubers, rhizomes, slips and seeds.

An “increase in yield” of a crop plant treated with the one or more fungal species includes an increase in fruit, grain or vegetative tissue production of the treated plant relative to that of a crop plant that is the same but which has not been treated with the one or more fungal species described herein when the treated and untreated plant are grown under the same growing conditions. An “untreated control plant” as used herein is a plant grown in a similar soil type under similar conditions (e.g., fertilizer application, watering, etc.) except that no fungal strain is applied to the plant. For example, an increase in yield of a treated wheat plant is an increase in the number and/or weight of wheat grains produced by the treated wheat plant relative to that of an untreated wheat plant grown under the same growth conditions. Typically, the increase in yield of a plant treated with the one or more fungal species is an increase in fruit, grain or vegetative tissue production of the treated plant relative to that of a healthy plant of the same type that has not been treated with the one or more fungal species described herein when the treated and untreated plant are grown under the same growing conditions. A healthy plant is a plant that is not infected with, or affected by, a plant pathogen. Typically, a healthy plant is a plant that is not infected with, or affected by, a plant pathogen, and which is grown under conditions for normal growth of that plant (e.g., is not under stress, such as nutrient or drought stress), such as, for example, the conditions under which the crop plant would be grown under during commercial crop production.

Increased yield in plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, chlorophyll content, nodulation, internode number and distance, root growth (e.g., root number, root length, root mass, root volume), shoot growth (e.g., shoot mass, leaf area, leaf number, plant height) seed size, fruit size, pod size, pod or ear number, seed number per pod or ear, seed mass, enhanced seed filling, reduced seed dispersal, reduced pod dehiscence and lodging resistance. As used herein, “agronomic benefits” means improving one or more of these factors thereby increasing the yield of the plant.

The crop plant may, for example, be one or more compatible crops selected from the group consisting of species of the genus Triticum, Glycine, Brassica, Gossypium, Zea, Corchorus, Saccharum, Medicago, Lolium, Cofea, Camellia, Oryza, Hordeum, Boehmeria, Nicotiana, Cannabis, oilseeds, grain legumes, vegetables, fruits, and/or combinations or hybrids thereof. It is contemplated that the list of the crop plants disclosed herein are mere examples for the skilled persons to understand the present disclosure. The crop plants may further include new future species and breeds as well as hybrids produced by grafting or transgenic species.

In preferred embodiments of the invention, the crop plant may, for example, be one or more crops selected from the group consisting of the species Triticum aestivum, Brassica napus, Brassica rapa, Brassica juncea, Gossypium hirsunum, Gossypium barbadense, Gossypium arboretum, Gossypium herbaceum, Zea mays, Medicago sativa, Lolium multiflorum, Corchorus capsularis, Saccharuim officinarum, Cannabis sativa, Coffea arabica, Coffea robusta, Camellia sinensis, Oryza sativa, Hordeum vulgare, Boehmeria nivea and Nicotiana tabacum.

In one embodiment, the crop plant is a cereal plant. Cereal plants include, for example, wheat, rice, corn (maize), canola, rye, oats, barley, sorghum, and some of the millets. In various embodiments, the crop plant is a cereal plant selected from the group consisting of wheat, rice and corn. In one aspect, the plant is a millet selected from the group consisting of finger millet (Eleusine coracana), foxtail millet (Setaria italica), browntop millet (Urochloa ramose), pearl millet (Pennisetum glaucum), Japanese millet/barnyard millet (Echinochloa esculenta), little millet (Panicum sumatrense), broomcorn millet/proso millet (Panicum miliaceum), Kodo millet (Paspalum scrobiculatum), fonio millet (Digitaria exilis), guinea millet (Brachiaria deflexa), great millet (Sorghum bicolor), Sonoran millet (Panicum hirticaule), Polish millet (Digitaria sanguinalis), adlay millet (Coix lacryma-jobi), and Taiwan oil millet (Spodiopogon formosanus).

In another embodiment, the crop plant is a fibre plant. Fibre plants include, for example, flax, hemp, jute, and cotton. In various embodiments, the crop plant is a fibre plant that is cotton.

In one embodiment, the crop plant is a legume. Legume plants include, for example, soybeans, alfalfa, clover, peanuts, lentils, lupins, peas, and chickpea. In various embodiments, the crop plant is a legume that is soybeans.

In some aspects, the crop plant is a cover crop. Cover crops include but are not limited to ryegrass, clover, red clover, white clover, crimson clover, annual medics, annual ryegrass. Italian ryegrass, canola, fine fescue, Kentucky bluegrass, orchard grass, and other grasses.

In other aspects, the crop plant is a grass such as switchgrass, tall fescue, meadow fescue, perennial ryegrass, Italian ryegrass, orchard grass, guinea grass, foxtail millet, pearl millet, Bahia grass and Miscanthus.

In embodiments of the invention, the plant is a “non-native” plant host of the fungal strain. “Non-native” plant host means that the fungi are heterologous to said plant insofar as the fungal strain was collected from a host other than said crop plant.

Endophytic fungi are known to have preferred hosts and growth conditions, and will not necessarily flourish, and therefore will not produce the desired stable SOC, in the absence of their typical growth environment or an association with their native hosts. Moreover, when considering the survival of the fungi in non-native plant hosts, it is difficult to anticipate whether the fungi will prove to be pathogenic to the non-native host. Therefore, fungal species may not readily be compatible with a non-native crop plant host.

### Inoculation

As used herein, the terms “inoculate,” “apply,” “treat,” and “deploy” are used interchangeably as are their associated nouns (i.e., “inoculation, “application,” “treatment” and “deployment”). The inoculation of the plant with the one or more fungal species may be achieved by any suitable means such as direct addition to the soil and/or plant roots and/or to soil proximal to plant roots, or may be achieved by an initial fungal inoculation of any propagation material, seeds, seedlings and/or immature plants of the crop plant prior to placement of the seed, seedling or immature plant in the soil within which the plant will grow. The inoculation of the one or more fungal species may also be achieved by direct addition to a cultivated soil prior to sowing seeds or planting seedlings that are coated or partially coated with one or more fungal species such that the fungi will become associated with, or grow proximal to, or grow into the roots of a crop plant as the crop matures.

By means of the inoculation, the fungi are deliberately encouraged to become established in the soil and/or grow proximal to, or grow into the roots of a plant (i.e., become associated with) that is a crop plant, wherein it would be understood the fungi may exist and grow in the soil or exist within the plant, or in both simultaneously. In aspects of the invention wherein the treatments or methods rely on the inoculation of a plant with a fungus, it would be understood the fungus need only be associated with the plant for parts of the fungus' lifecycle and that the fungus may survive in the soil in the absence of a plant host or host crop plant.

In other embodiments, the inoculation may be considered a semi-permanent inoculation to a plot of soil that is cultivated, such that the fungus is deployed to said plot of soil and is retained by the soil as the crops are rotated, even in the absence of crops for periods of time.

In some embodiments, the soil is inoculated with the one or more fungal species. The soil may be inoculated with the one or more fungal species prior to planting the plant, for example before, during, or after tilling the soil in preparation for planting. In other embodiments, the soil may be inoculated with the one or more fungal species after the plant has been planted. In some embodiment, the soil is inoculated with the one or more fungal species by planting in the soil plants that have been inoculated with the one or more fungal species.

In some embodiments, the step of inoculating a crop plant comprises applying the one or more fungal species to seeds of the plant prior to planting.

In some embodiments, the step of inoculating a crop plant comprises applying the one or more fungal species to seedlings of the plant.

In some embodiments, the step of inoculating soil comprises deploying the one or more fungal species to a plot of soil that is cultivated, such that the fungus is retained by the soil as the crops are rotated, even in the absence of crops for periods of time.

In one embodiment, the plants are inoculated with one or more fungal species as a seed coating before, during or after one or more of the stages of germination of a seed, or as a root inoculant of a seedling. For example, the treatment may be applied as a seed coating to seeds en masse prior to sowing a crop.

The one or more fungal species for inoculation may be in any suitable form, including, for example, as hyphae, mycelia, conidia and/or combinations thereof. In general, the one or more fungal species for inoculating the plant will be in a form that is substantially free of contaminating microorganisms, with the exception that additional desirable microbes may be added for additional benefits.

In one aspect, there is provided a soil for increasing yield of a crop plant, the soil comprising one or more fungal species having a nuclear ribosomal internal transcribed spacer 2 (ITS2) sequence that is at least 90% identical to the nucleotide sequence of any one of SEQ ID NOs: 1-11.

In some embodiments, the inoculant may be in the form of a dried powder, a spray, a slurry, a sachet, a liquid, a jelly, a seed coating, an enhancer, and/or combinations thereof.

In some embodiments, the inoculant is in the form of a seed coating, a foliar spray, granule, powder, soil drench or a root dip.

In one embodiment, the inoculant is in the form of a seed coating.

In one embodiment, the inoculant is in the form of a foliar spray.

In one embodiment, the inoculant is in the form of a root dip.

In one embodiment, the inoculant is a granule.

In one embodiment, the inoculant is a powder.

In one embodiment, the composition is a soil drench.

In some embodiments, the one or more fungal species are compatible with commonly used agricultural fungicides. “Compatible” means the one or more fungal species in the treatment is not killed or substantially inhibited (growth or germination or otherwise) by the fungicide, thereby allowing the fungi in the treatment to flourish while restricting the growth of undesirable fungal strains that may have a deleterious effect on the soil; the proximal crops or plants, and/or the level of carbon sequestration and stable carbon production. The fungicide may be any synthetic or natural compound that has a fungistatic or fungicidal function and are commonly used in agriculture. Based on their mode of action, they may kill the fungi or inhibit the germination of fungal spores.

The composition and/or inoculant may comprise suitable solid or liquid carriers and/or adhesive agents.

Suitable solid carriers include mineral earths (e.g., calcium phosphate, calk, clay, diatomaceous earth, dolomite, kaolin, silicates, silica gels, talc, etc), cellulose, and starch. Suitable liquid carriers include water, or any other liquid solvents which are not toxic to the fungus or the plant.

The composition may be prepared in a known manner, by mixing it with customary adjuvants, such as, for example, customary extenders and also solvents or diluents, colorants, wetters, dispersants, emulsifiers, antifoams, preservatives, secondary thickeners, stickers, and also water.

Colorants which may be present in a seed-dressing composition which can be used in accordance with the invention include all colorants which are customary for such purposes. In this context it is possible to use not only pigments, which are of low solubility in water, but also water-soluble dyes. Examples include the colorants known under the designations Rhodamine B, C.I. Pigment Red 112 and C.I. Solvent Red 1.

Wetters that may be present in the seed-dressing composition include all of the substances which promote wetting and which are customary in the formulation of active agrochemical ingredients Use may be made preferably of alkylnaphthalenesulphonates, such as diisopropyl- or diisobutyl-naphthalenesulphonates.

Dispersants and/or emulsifiers which may be present in the seed-dressing composition include all of the nonionic, anionic and cationic dispersants that are customary in the formulation of active agrochemical ingredients, Use may be made preferably of nonionic or anionic dispersants or of mixtures of nonionic or anionic dispersants. Suitable nonionic dispersants are, in particular, ethylene oxide-propylene oxide block polymers, alkylphenol polyglycol ethers and also tristyrylphenol polyglycol ethers, and the phosphated or sulphated derivatives of these. Suitable anionic dispersants are, in particular, lignosulphonates, salts of polyacrylic acid, and arylsulphonates-formaldehyde condensates.

Antifoams which may be present in the seed-dressing composition include all of the foam inhibitors that are customary in the formulation of active agrochemical ingredients. Use may be made preferably of silicone antifoams and magnesium stearate.

Preservatives which may be present in the seed-dressing composition include all of the substances which can be employed for such purposes in agrochemical compositions. Examples include dichlorophen and benzyl alcohol hemiformal.

Secondary thickeners which may be present in the seed-dressing composition include all substances which can be used for such purposes in agrochemical compositions. Those contemplated with preference include cellulose derivatives, acrylic acid derivatives, xanthan, modified clays and highly disperse silica.

Stickers which may be present in the seed-dressing composition include all customary binders which can be used in seed-dressing products. Preferred mention may be made of polyvinylpyrrolidone, polyvinyl acetate, polyvinyl alcohol and tylose.

### Soil Organic Carbon

The fungi used in the methods described herein will generally be capable of sequestering and fixing carbon from atmospheric carbon dioxide and converting this carbon to complex polysaccharides for storage as stable carbon in the soil. The sequestered and fixed carbon may also be converted and stored as a stable carbon source by the fungi in the fungi itself as, for example, melanin, chitin, lignin, suberin and carotenoid compounds, or the fungi may exude these compounds to increase the stable carbon in the soil. The deployed fungal endophyte may also convert simple polysaccharide exudate from a host plant into complex polysaccharides for storage as stable carbon in the soil, or within the fungi itself. Lastly, the stability of organic carbon may be enhanced in soil with more stable soil aggregates.

The methods and treatments of the present invention may increase the overall levels of carbon in the soil, but even in cases where overall carbon remains the same or is only slightly increased, it would be understood that the levels of stable carbon in the soil may be increased due to the production and exudation in the soil of complex polysaccharides by the disclosed fungal species.

The fungi may be particularly useful to increase overall levels of carbon in the soil and/or levels of stable carbon in the soil where the soil has a soil organic carbon (SOC) level below a particular threshold. In some aspects, the threshold is a SOC level (% wt/wt) below 5%, 4%, 3%, 2%, 1%, 0.9%, 0.8%, 0.7%, 0.6%, 0.5%, 0.4%, 0.3%, 0.2%, or 0.1%.

The increase in overall soil carbon and stable soil carbon of a soil that is subjected to the treatments and/or methods of the present disclosure compared to an untreated control (i.e., a “non-inoculated control soil”) may be quantified by any methods known to those skilled in the art. The control would be a similar soil sample that had not been exposed to an endophytic fungus as claimed herein (i.e., a fungus had not been deployed in the soil or associated with a plant that had been cultivated in said soil). “Similar soil sample” means that the soil would be from a proximal area with a similar climate and, if the soil had been cultivated, the control sample would have been cultivated by the same plant as the test soil.

In one embodiment, an increase in soil organic carbon is an increase in stable carbon. An increase in the sequestration of atmospheric carbon for storage as stable carbon in the soil and increasing the levels of stable carbon in the soil, is an increase relative to the amount of sequestration of atmospheric carbon for storage as stable carbon in the soil, and levels of stable carbon in the soil, produced by a plant that has not been treated with the methods of the present disclosure.

Application of the fungi to the plant and/or soil may have one or more desirable effects on the soil and/or associated crops cultivated in the treated soil, including for example, sequestering atmospheric carbon for storage as stable carbon in the soil; and/or increasing the levels of stabilised carbon in the soil.

The inoculation of the soil and/or plants with the fungi may have simultaneous beneficial effects on the soil. For example, sequestration of atmospheric carbon by endophytic fungi as described herein can lead to an increase in the complex polysaccharides in the soil resulting in long-term storage of sequestered atmospheric carbon in a stable form.

Soil organic carbon is the overall soil carbon content of a soil and may be also generally referred to as total organic carbon (TOC) (the terms may be used interchangeably), and this refers only to the carbon component of the organic matter in the soil. However, fluctuations in soil organic carbon may not necessarily correlate to the same fluctuations in stable soil carbon. Indeed, soils subjected to the treatments and methods may demonstrate minimal increases in TOC, but the percentage of said TOC that is captured in a stable form in the soil or in the fungi proliferating in the soil (i.e., complex polysaccharides, melanin, chitin, lignin, suberin and carotenoid compounds) may increase. The skilled addressee would also understand that changes in TOC and stable carbon in soil as a result of the treatments and methods of the present may take weeks, months or years, and therefore appropriate measurement timeframes must be applied. In one embodiment, the increase in soil organic carbon in a soil comprises an increase in stable carbon in the soil.

The soil carbon may be measured by methods including, but not limited to, dry combustion or elemental tests that may be analysed using, for example, the LECO method, and loss on ignition (LOI) tests that may be analysed using the Walkley-Black method (see, for example, Walkley A, and Black IA (1934) An examination of the Degtjareff method for determining soil organic matter, and a proposed modification of the chromic acid titration method. Soil Science 37, 29-38). To assess the prevalence of different types of carbon on the TOC (i.e., to measure the stable, or “recalcitrant” organic carbon), methods may be employed to fractionate to TOC by, for example, measuring soil respiration or the bulk density of the soil.

In some embodiments of the invention, the fungal inoculation of soil and/or the plant results in an increase in soil aggregate stability. The increase in soil aggregate stability, or soil aggregation per se, of a soil that is subjected to the treatments and/or methods described herein compared to a control may be quantified by any methods known to those skilled in the art. The control would be a similar soil sample that had not been exposed to the relevant fungus (i.e., a fungus had not been deployed in the soil or associated with a plant that had been cultivated in said soil). “Similar soil sample” means that the soil would be from a proximal area with a similar climate and, if the soil had been cultivated, the control sample would have been cultivated by the same plant as the test soil. The soil aggregate stability may be quantified by measurements compared to controls such as, but not limited to, soil mean weight diameter (MWD), geometric mean diameter (GMD), fractal dimension (D), percentage of aggregates destruction (PAD) and water-stable aggregates stability rate (WSAR). An increase in the MWD, GMD, WSAR and D values are indicative of an increase in soil aggregate stability, while a decrease in PAD value is indicative of an increase in soil aggregate stability.

In various embodiments of the invention, the fungal inoculation may have one or more desirable effects on the soil and/or associated crop plants cultivated in the treated soil, including, but not limited to, sequestering atmospheric carbon for storage as stable carbon in the soil; providing agronomic benefits to the crop plants; increasing the levels of stabilised carbon in the soil used to cultivate the crop plants; and/or increasing the soil aggregate stability of the soil used to cultivate crop plants. In other embodiments of the invention, the fungal inoculation may have two or more of the aforementioned desirable effects on the soil and/or associated crop plants cultivated in the treated soil, or three or more of the aforementioned desirable effects on the soil and/or associated crop plants cultivated in the treated soil.

That the fungal inoculation of the methods of the invention may have numerous, simultaneous effects on the soil and/or associated crop plants cultivated in the treated soil is, in part, possible because some of the desirable effects contribute to other desirable effects. For example, increasing soil aggregate stability is related to the enhanced (and/or longer-term) storage of sequestered atmospheric carbon as well as providing agronomic benefits to said crop plants by virtue of stably aggregated soil being more productive through, for example, improved water retention. In another example, sequestration of atmospheric carbon by the melanised fungi as described herein can lead to an increase in the complex polysaccharides in the soil resulting in long-term storage of sequestered atmospheric carbon in a stable form,

### Fractionation of Soil Organic Carbon

In certain aspects, the disclosed mixtures increase organic carbon in the soil. Soil organic C (SOC) concentration in mineral soils (0-10 cm depth) varies from <0.2% to ≥1.6% C; above this concentration, a soil is classified as a ‘peat’ soil, for example, in peatlands or tundra lands.

Soil organic C in mineral soils contains a range of organic substances at various stages of decomposition such as plant materials both produced aboveground (straw, litter) and belowground (roots, root exudates), fungal hyphae, soil fauna, and microbial biomass and their products. Organic compounds include lipids, proteins, carbohydrates, quinones, and their derivatives. Major functional groups include alkyl C (10-45 ppm), N-alkyl and methoxy C (45-60 ppm), O-alkyl C (60-110 ppm), aromatic C (110-145 ppm), phenolic C (145-165 ppm), and amide and carboxyl C (165-215 ppm), as identified in ¹³C NMR spectra of SOC (Almeida et al. 2021). These are also grouped as aromatic, aliphatic and polysaccharide groups.

Since SOC consists of different C substances, which turnover (decompose) at different rates, persist in soil for different periods, stabilize with minerals with different mechanisms, and contribute to bio-physico-chemical functions in separate ways (Chenu et al. 2015). Most components of SOC are separated by chemical oxidation (acids, alkali, oxidants), biological (decomposition rates, microbial respiration), and physical methods. Of these, physical methods are preferred because these methods cause minimum disturbance, disruption, and alteration of SOC substances in soil. The physical methods are based on density, size, and sedimentation of soil, and justification of these methods are given by Poeplau et al. (2018).

Density, size, and sedimentation procedures broadly fractionate SOC or SOM in three groups, fPOC or fPOM, aggregate occluded particulate organic C (oPOC, or oPOM) and silt+clay size associated or fine mineral-associated organic C (MAOC or MAOM). Dissolved organic C, DOC or DOM are also measured and plays a significant role in MAOM formation although it accounts for <2% of total Plant C or SOC.

The stabilized MAOM are separated from labile organic matter using density and size fractionation procedure (Poeplau et al. 2018; Mayer et al. 2022; Rodrigues et al. 2022), The organic matter that floats in the heavy density liquid, either sodium iodide (NAI) solution or sodium polytungstate (SPT, Na₆[H₂W₁₂O₄₆] or 3Na₂WO₄·9WO·H₂O) at 1.8 Mg m⁻³ (1.8 g cm⁻³) or <1.8 Mg m⁻³ density soil organic matter are considered as fPOM. After separation of fPOM, occluded particulate organic matter within aggregates, oPOM and MAOM are usually dispersed either using ultrasonic energy or sodium hexametaphosphate (Na₆[(PO₃)₆] solution to disperse the soil particles. In the former, ultrasonic energy, ~400-500 J mL⁻¹ is applied to the soil in the SPT solution (1.8 Mg m⁻³) contained in a temperature-controlled container. Excess oPOM is separated, and the remaining soil is sieved through 53 μm sieve to collect <53 μm as MAOM. The >53 μm fraction remained on the sieve is considered as the organic matter in the sand-size fraction, usually contains small amount of organic C and could be added to the fPOM fraction. This is circumvented when after separation of fPOM, excess SPT is washed from the remaining soil and the soil is dispersed in sodium hexametaphosphate and sieved through 53 μm sieve to collect <53 μm as MAOM. The >53 μm fraction remained on the sieve is considered as the organic matter occluded in the sand-size aggregates or aggregate C since sand fraction as such contains only small amount of organic C. Thus, SOC is separated into three fractions: fPOM, oPOM, and MAOM. Obviously, dissolved organic matter (DOM) is either lost or added to the MAOM fraction although in most mineral soils, DOM constitutes <2% of SOC (Poeplau et al. 2018). For practical purposes and routine SOC fractionation, SOM can be separated into >53 μm POM (fPOM+oPOM) and <53 μm MAOM (Lavallee et al. 2019).

The MAOM fraction provides the long-term storage of SOC (Kleber et al. 2015; Hemingway et al. 2019). However, MAOM is subject to the C saturation of fine silt+clay (<53 μm) or fine mineral fraction, which is dependent on silt+clay contents (Feng et al, 2013) and their mineralogy, Fe and Al (hydro-)oxides, specific surface area, soil architecture, nature of organic C inputs, especially their C and N contents, and soil pH. Once the C saturation of mineral fraction is achieved, further SOC sequestration for the long-term storage as MAOM is not likely to occur although the potential turnover through C mineralization and fresh C addition may still be required (Mayer et al, 2022; Rodrigues et al, 2022). From the boundary line approach, Feng et al. (2013) estimated that the silt+clay size fraction (<2 μm) may store 84±1 g C kg-1 silt+clay size fraction in 2:1 clay dominant (smectite, illite, vermiculite) soil, and 43±1 g C kg-1 silt+clay in 1:1 clay dominant (kaolinite) soil. This provides a ‘rule of thumb’ estimate to identify a soil, in which SOC may be sequestered long-term in the MAOM fraction. It is worth noting here that the 20-53 μm MAOM fraction may contain silt-size micro-aggregates which may have faster turnover rate than the <20 μm MAOM.

Further, organic carbon may not uniformly cover the surface of the fine mineral fraction. For example, Schweizer et al. (2021) found that clay surfaces of the soil containing low clay contents (5-18%) had twice as much organic C in the MAOM than the high clay soils.

It is sobering to note that long-term field experiments have shown that it is less likely that SOC will be sequestered in the stabilized MAOM fraction if this fraction is already saturated (Mayer et al. 2022; Rodrigues et al., 2022), and further C inputs will be stabilized in the oPOM fraction (occluded in aggregates) or remain fPOM. However, oPOM is readily lost when the soil is disturbed. It is not known whether there is a saturation limit for the oPOM fraction, fPOM can be increased in soil indefinitely although this fraction is affected by global warming, and quantity and quality of continuous C inputs more than the other SOM fractions (Lugato et al. 2021; Rocci et al. 2021).

SOC fractionation protocols vary widely. Poeplau et al (2018) compared two protocols in use at the time of the study. Details of these protocols and a preferred protocol are given by Poeplau et al. (2018). In conclusion, they found that no SOC fraction identified the rapid turnover rate component, that particle-size separation was better for separating the fPOM than that the oPOM incorporated into aggregates, and the separation of silt+clay-size fraction from the sand-size fraction was the most effective protocol in identifying fractions of different turnover rates. Admittedly, since microbial inoculants in the rhizosphere may be involved in aggregation (Mugerwa and McGee 2017, Buss et al. 2021), and, therefore, soil structure, it is recommended that in such situations oPOM separation may be desirable to detect the effect of inoculants in C accumulation in the rhizosphere of the microbial inoculated plants. However, as stated above, since turnover rate of oPOM is uncertain, and subject to disturbance, it should not be considered for long-term C sequestration in soil.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

All headings are for the convenience of the reader and should not be used to limit the meaning of the text that follows the heading, unless so specified.

The present invention is further illustrated by the following examples that should not be construed as limiting. The contents of all references, patents, and published patent applications cited throughout this application, as well as the Figures, are incorporated herein by reference in their entirety for all purposes. In order to exemplify the nature of the present invention such that it may be more clearly understood, the following non-limiting examples are provided.

### EXAMPLES

### Example 1. Fungal Strains are Capable of Phosphate Solubilization

Phosphorus is essential for the growth and productivity of plants. It plays an important role in plants in many physiological activities such as cell division, photosynthesis, development of a healthy root system, and utilization of carbohydrates. Phosphorus (P) has long been considered the second most limiting nutrient for plant growth in terrestrial ecosystems after nitrogen (N). Organic P is often the dominant form of P found in soils and may constitute up to 90% of the total P in soil (Sharma et al 2013). P mineralisation is a prerequisite to converting organic P into a plant available form. Microbes that have the ability to solubilize phosphate can help improve plant growth when growers opt for less soluble forms of phosphate in fertilizer.

The ability of fungal strains to solubilize P was tested in vitro by plating on solid Pikovskaya (PVK) media containing insoluble superphosphate (Doilom et al 2020). Strains with a clear zone of dissolved phosphate in solid Pikovskaya medium indicate the presence of phosphate solubilizing ability.

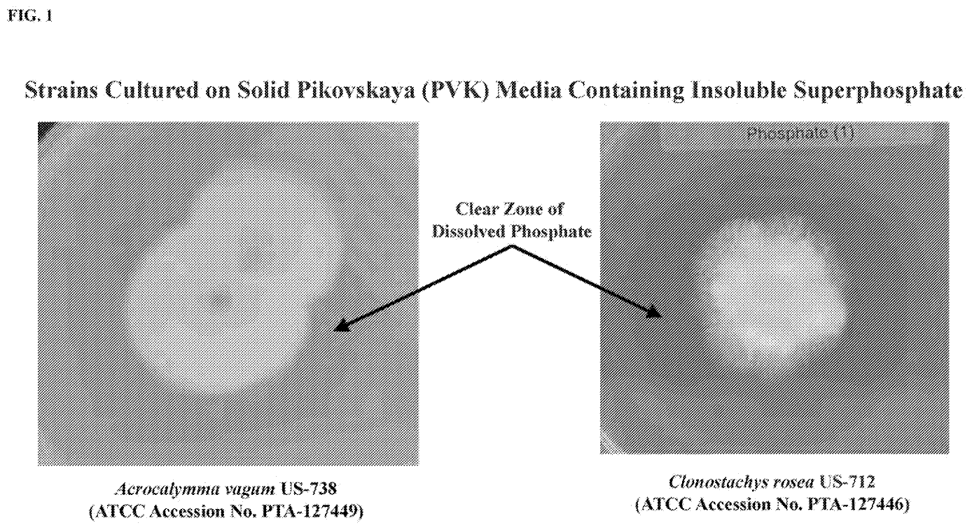

Acrocalymma vagum US-738 (ATCC Accession No. PTA-127449), Acrocalymma vagum US-445 (ATCC Accession No. PTA-127444), Leptodontidium orchidola US-210 (ATCC Accession No. PTA-127441), Leptodontidium orchidola US-70 (ATCC Accession No. PTA-127439), Clonostachys rosea US-114 (ATCC Accession No. PTA-127443), Clonostachys rosea US-712 (ATCC Accession No. PTA-127446). Trichoderma hamatum US-724 (ATCC Accession No PTA-127448), and Trichoderma longipile/spirale TS-77 (ATCC Accession. No. PTA-127440) each produced a clear zone of dissolved phosphate in solid Pikovskaya medium demonstrating their ability to solubilize phosphate. Representative images with Acrocalymma vagum US-738 (ATCC Accession No. PTA-127449) and Clonostachys rosea US-712 (ATCC Accession No. PTA-127446) are presented in FIG. 1.

### Example 2. Leptodontidium orchidola US-70 (ATCC Accession No. PTA-127439) and Periconia macrospinosa AU-7083 (NMI Accession No. V22/019796) Produce Siderophores

Siderophores are low molecular weight organic iron (and other metals) coordination compounds produced by some species of bacteria and fungi. Siderophores produced by root-associated fungus may enhance plant growth by increasing the availability of Fe near the root (Hider and Hong 2010). The siderophore production ability of microorganisms can be followed and compared over time using the CAS method (Alexander and Zuberer 1991). This assay is based on a competition for iron between the ferric complex of an indicator dye, chrome azurol S (CAS), and putative siderophores produced by the microorganism. The iron is removed from the medium by the siderophore, which has a higher affinity than CAS for iron(III). The positive reaction results in a colour change from blue to orange of the CAS reagent, which indicates siderophore production.

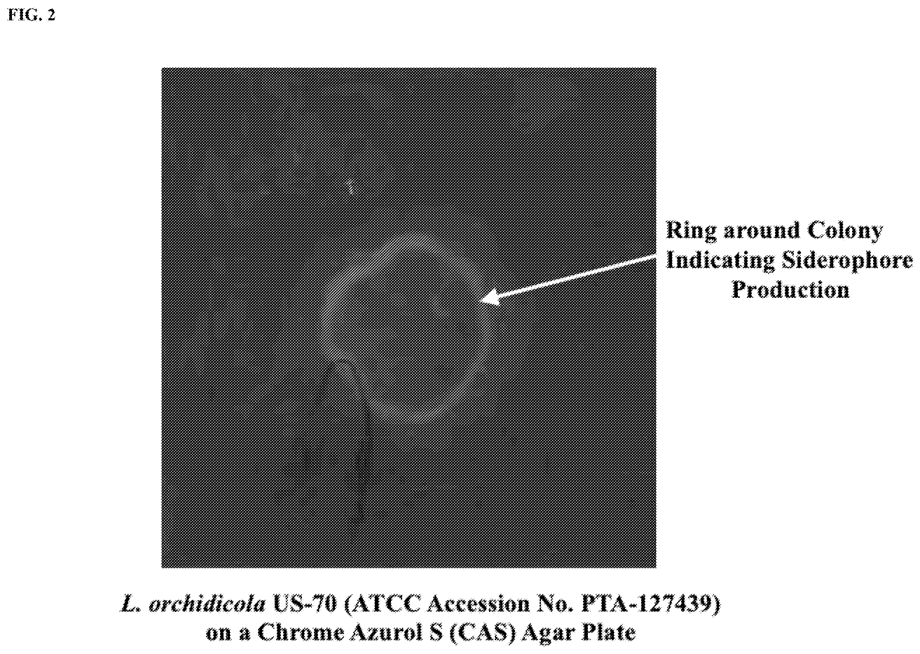

Leptodontidium orchidola US-70 (ATCC Accession No. PTA-127439) was cultured on an agar plate containing CAS and produced a distinct ring around the fungal colony demonstrating the ability of the strain to produce siderophores (see FIG. 2). Periconia macrospinosa AU-7083 (NMI Accession No. V22/019796) produced a similar ring on agar plates containing CAS indicating an ability to produce siderophores (data not shown).

### Example 3. Periconia macrospinosa AU-7083 (NMI Accession No. V22/019796) and Periconia macrospinosa DMTR-CTR-1852 (NMI Accession No. V22/006358) Applied to Wheat Increase Soil Carbon

Periconia macrospinosaAU-7083 (NMI Accession No. V22/019796) and Periconia macrospinosa DMTR-CTR-1852 (aka AU-1852) (NMI Accession No. V22/006358) were applied to wheat seeds via liquid injection (LI) or biopriming (BP). Condo and Bennett varieties of wheat seeds were evaluated. 1 mL of fungal suspension per seed was applied at planting for LI application. Wheat seeds were soaked in the fungal suspension for 24 hours at room temperature for BP application. The fungal suspension was at a concentration of about 1×10⁴ CFU/mL for both application methods.

The wheat seeds were planted in non-sterile soil and grown in a glasshouse for 8 weeks at an ambient temperature ranging from 15° C. to 30° C. Soil carbon measurements were taken at the end of the growth period. Soil samples were dried and weighed, and total carbon was measured by combustion using the LECO® carbon analyser model C832. Soil carbon from treated plants was compared to that from untreated plants. Each measurement represents the average of at least three replicates.

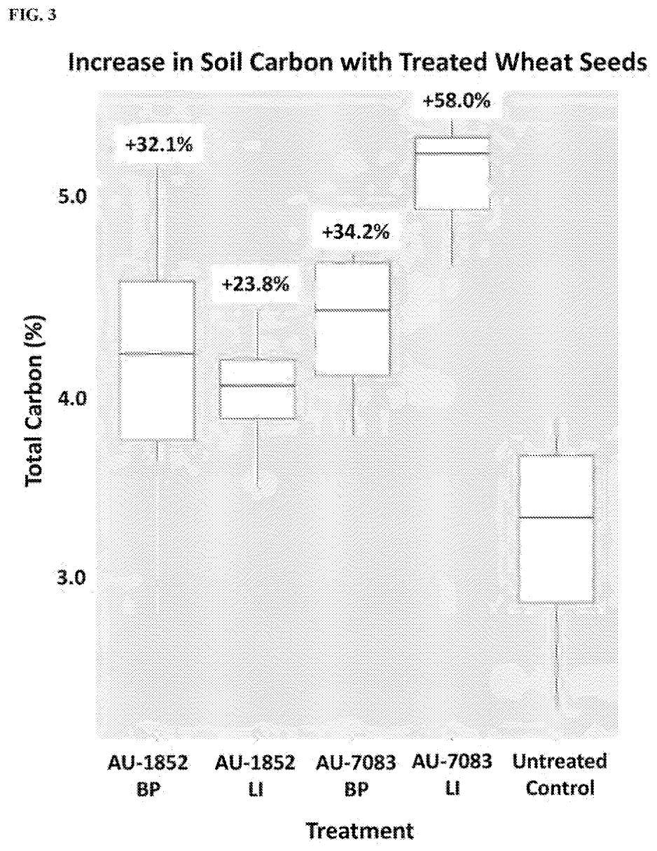

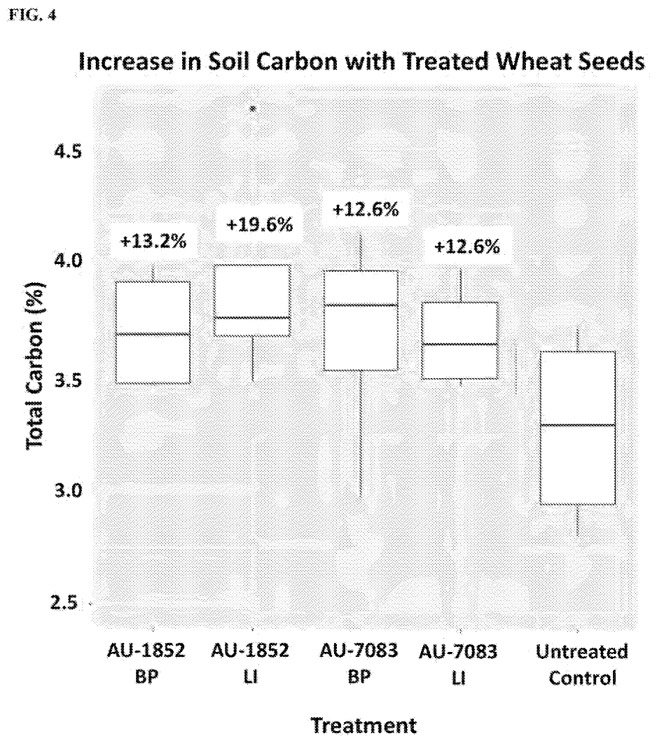

Soil carbon increased between 23.8% and 58.0% over untreated control in Bennett wheat seeds treated with Periconia macrospinosa AU-7083 (NMI Accession No. V22/019796) or Periconia macrospinosa DMTR-CTR-1852 (NMI Accession No. V22/006358) (see FIG. 3). Similarly, soil carbon was between 12.6% and 19.6% greater with Condo wheat seeds treated with Periconia macrospinosa AU-7083 (NMI Accession No. V22/019796) or Periconia macrospinosa DMTR-CTR-1852 (NMI Accession No. V22/006358) (see FIG. 4).

### Example 4. Leptodontidium orchidola US-210 (ATCC Accession No. PTA-127441)

Applied to Soybeans Increases Soil Carbon and Crop Yield Soybean seeds were planted in potted soil in a greenhouse. For treated plants, at the time of sowing an agar plug colonized with Leptodontidium orchidola US-210 (ATCC Accession No. PTA-127441) was placed with the fungal cells contacting the soybean seeds. The temperature in the greenhouse was maintained at 24° C. to 28° C. with a 16-hour day length. Plants were watered manually once daily. Plants were grown to full maturity at which time the total organic carbon in the soil and the crop yields were measured with four replicates for each treatment group.

Application of Leptodontidium orchidola US-210 (ATCC Accession No. PTA-127441) to soybeans increased both total organic carbon and yield (see Tables 3 and 4).

TABLE 3

| | Total Organic | % Change |
|---|---|---|
| | Carbon | Compared to |
| Treatment | (TOC) (%) | Untreated Control |
| Untreated Control | 2.2391619 | — |
| Leptodontidium orchidicola | 2.2854083 | +2.02% |
| US-210 (ATCC Accession No. | | |
| PTA-127441 | | |

TABLE 4

| | | % Change to |
|---|---|---|
| | | Compared |
| | | Untreated |
| Treatment | Yield (g) | Control |
| Untreated Control | 11.196358 | — |
| Leptodontidium orchidicola | 11.605471 | +3.53% |
| US-210 (ATCC Accession No. | | |
| PTA-127441) | | |

### Example 5. Leptodontidium orchidola US-210 (ATCC Accession No. PTA-127441) Applied to Corn Increases Soil Carbon and Crop Yield

### Background

To evaluate the effects of Leptodontidium orchidola US-210 (ATCC Accession No. PTA-127441) on soil carbon and crop yield, a corn field trial was conducted near Olivia, 1.0 Minnesota, in the United States.

### Materials and Methods

Leptodontidium orchidola US-210 (ATCC Accession No. PTA-127441) was applied directly to corn seeds at a rate of about 300 CFU per seed immediately prior to sowing with a neutral carrier Untreated corn seeds were sown as a control, Six replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the ratio of TOC at harvest (% wt/wt) to the TOC at baseline (% wt/wt). The percent change between treated and untreated groups was calculated by subtracting the mean carbon build of the untreated group from that of the treated group.

### Results

Results for TOC and yield are shown in Tables S and 6, respectively.

TABLE 5

| | Mean Carbon | % Change to |
|---|---|---|
| | Build | Compared |
| | (TOCₕₐᵣᵥₑₛₜ/ | Untreated |
| Treatment | TOC_{baseline}) | Control |
| Untreated Control | 1.006631133 | — |
| Leptodontidium orchidicola | 1.071469032 | +6.48% |
| US-210 (ATCC Accession No. | | |
| PTA-127441) | | |

TABLE 6

| | | % Change |
|---|---|---|
| | | Compared to |
| | Yield | Untreated |
| Treatment | (Bushels/Acre) | Control |
| Untreated Control | 212.95 | — |
| Leptodontidium orchidicola | 221.11 | +1.04% |
| US-210 (ATCC Accession | | |
| No. PTA-127441) | | |

Leptodontidium orchidola US-210 (ATCC Accession No. PTA-127441) increased both soil carbon and yield compared to the untreated control with corn.

### Example 6. Leptodontidium orchidola US-210 (ATCC Accession No, PTA-127441) Applied to Spring Wheat Increases Soil Carbon and Crop Yield

### Background

To evaluate the effects of Leptodontidium orchidola US-210 (ATCC Accession No. PTA-127441) on soil carbon and crop yield, a spring wheat field trial was conducted near American Falls, Idaho, in the United States.

### Materials and Methods

Leptodontidium orchidola US-210 (ATCC Accession No. PTA-127441) was applied directly to spring wheat seeds at a rate of about 100 CFU per seed immediately prior to sowing with a neutral carrier. Untreated spring wheat seeds were sown as a control, Five replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the ratio of TOC at harvest (% wt/wt) to the TOC at baseline (% wt/w). The percent change between treated and untreated groups was calculated by subtracting the mean carbon build of the untreated group from that of the treated group.

### Results

Results for TOC and yield are shown in Tables 7 and 8, respectively.

TABLE 7

| | Mean | % Change |
|---|---|---|
| | Carbon Build | Compared to |
| | (TOCₕₐᵣᵥₑₛₜ/ | Untreated |
| Treatment | TOC_{baseline}) | Control |
| Untreated Control | 1.097364745 | — |
| Leptodontidium orchidicola | 1.117943225 | +2.06% |
| US-210 (ATCC Accession No. | | |
| PTA-127441) | | |

TABLE 8

| | | % Change |
|---|---|---|
| | | Compared to |
| | Yield | Untreated |
| Treatment | (Bushels/Acre) | Control |
| Untreated Control | 127.94 | — |
| Leptodontidium orchidicola | 129.88 | +1.52% |
| US-210 (ATCC Accession No. | | |
| PTA-127441) | | |

Leptodontidium orchidola US-210 (ATCC Accession No. PTA-127441) increased both soil carbon and yield compared to the untreated control with spring wheat.

### Example 7. Trichoderma longipile/spirale US-77 (ATCC Accession No. PTA-127440) Applied to Spring Wheat Increases Soil Carbon and Yield

### Background

To evaluate the effects of Trichoderma longipile/spirale US-77 (ATCC Accession No. PTA-127440) on soil carbon and yield, a spring wheat field trial was conducted near Olivia, Minnesota, in the United States.

### Materials and Methods

Trichoderma longipile/spirale US-77 (ATCC Accession No. PTA-127440) was applied directly to spring wheat seeds at a rate of about 100 CFU per seed immediately prior to sowing with a neutral carrier. Untreated soybean seeds were sown as a control. Five replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the ratio of TOC at harvest (% wt/wt) to the TOC at baseline (% wt/wt). The percent change between treated and untreated groups was calculated by subtracting the mean carbon build of the untreated group from that of the treated group.

### Results

Results for TOC and yield are shown in Tables 9 and 10, respectively.

TABLE 9

| | Mean | % Change |
|---|---|---|
| | Carbon Build | Compared to |
| | (TOCₕₐᵣᵥₑₛₜ/ | Untreated |
| Treatment | TOC_{baseline}) | Control |
| Untreated Control | 0.9947019008 | — |
| Trichoderma longipile spirale | 1.016737075 | +22.04% |
| US-77 (ATCC Accession No. | | |
| PTA-127440) | | |

TABLE 10

| | | % Change |
|---|---|---|
| | | Compared to |
| | Yield | Untreated |
| Treatment | (Bushels/Acre) | Control |
| Untreated Control | 45.03 | — |
| Trichoderma longipile spirale | 50.04 | +11.13% |
| US-77 (ATCC Accession No. | | |
| PTA-127440) | | |

Trichoderma longipile/spirale US-77 (ATCC Accession No. PTA-127440) increased both soil carbon and yield compared to the untreated control with spring wheat.

### Example 8. Trichoderma longipile/spirale US-77 (ATCC Accession No. PTA-127440) Applied to Soybean Increases Soil Carbon

### Background

To evaluate the effects of Trichoderma longipile/spirale US-77 (ATCC Accession No, PTA-127440) on soil carbon, a soybean field trial was conducted near Sanborn, Iowa, in the United States.

### Materials and Methods

Trichoderma longipile/spirale US-77 (ATCC Accession No. PTA-127440) was applied directly to soybean seeds at a rate of about 1000 CF U per seed immediately prior to sowing with a neutral carrier. Untreated soybean seeds were sown as a control. Six replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the ratio of TOC at harvest (% wt/wt) to the TOC at baseline (% wt/wt). The percent change between treated and untreated groups was calculated by subtracting the mean carbon build of the untreated group from that of the treated group.

### Results

Results for TOC are shown in Table 11. The yield observed with the treated and untreated control plants was not statistically different.

TABLE 11

| | Mean | % Change |
|---|---|---|
| | Carbon Build | Compared to |
| | (TOCₕₐᵣᵥₑₛₜ/ | Untreated |
| Treatment | TOC_{baseline}) | Control |
| Untreated Control | 1.108787504 | — |
| Trichoderma longipile spirale | 1.126208002 | +1.74% |
| US-77 (ATCC Accession No. | | |
| PTA-127440) | | |

Trichoderma longipile/spirale US-77 (ATCC Accession No. PTA-127440) increased soil carbon compared to the untreated control with soybeans.

### Example 9. Acrocalymma vagum US-445 (ATCC Accession No. PTA-127444) Applied to Corn Increases Soil Carbon and Yield

### Background

To evaluate the effects of Acrocalymma vagum US-445 (ATCC Accession No. PTA-127444) on soil carbon, a corn field trial was conducted near Greenville, Mississippi, in the United States.

### Materials and Methods

Acrocalymma vagum US-445 (ATCC Accession No, PTA-127444) was applied directly to corn seeds at a rate of about 1000 CFU per seed immediately prior to sowing with a neutral carrier. Untreated corn seeds were sown as a control. Five replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the ratio of TOC at harvest (% wt/wt) to the TOC at baseline (% wt/wt). The percent change between treated and untreated groups was calculated by subtracting the mean carbon build of the untreated group from that of the treated group.

### Results

Results for TOC are shown in Tables 12. The yield observed with the treated and untreated control plants was not statistically different.

TABLE 12

| | Mean | % Change |
|---|---|---|
| | Carbon Build | Compared to |
| | (TOCₕₐᵣᵥₑₛₜ/ | Untreated |
| Treatment | TOC_{baseline}) | Control |
| Untreated Control | 0.9307062926 | — |
| Acrocalymma vagum | 1.113971059 | +18.33% |
| US-445 (ATCC Accession No. | | |
| PTA-127444) | | |

Acrocalymma vagum US-445 (ATCC Accession No. PTA-127444) increased soil carbon compared to the untreated control with corn.

### Example 10. Acrocalymma vagum US-738 (ATCC Accession No. PTA-127449) Applied to Corn Increases Soil Carbon and Yield

### Background

To evaluate the effects of Acrocalymma vagum US-738 (ATCC Accession No. PTA-127449) on soil carbon and yield, a corn field trial was conducted near Greenville, Mississippi, in the United States.

### Materials and Methods

Acrocalymma vagum US-738 (ATCC Accession No. PTA-127449) was applied directly to corn seeds at a rate of about 1000 CFU per seed immediately prior to sowing with a neutral carrier. Untreated corn seeds were sown as a control. Five replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the Catio of TOC at harvest (% wt/wt) to the TOC at baseline (% wt/wt). The percent change between treated and untreated groups was calculated by subtracting the mean carbon build of the untreated group from that of the treated group.

### Results

Results for TOC and yield are shown in Tables 13 and 14, respectively.

TABLE 13

| | Mean Carbon Build | % Change Compared |
|---|---|---|
| Treatment | (TOCₕₐᵣᵥₑₛₜ/TOC_{baseline}) | to Untreated Control |
| Untreated Control | 0.9307062926 | — |
| Acrocalymma vagum | 0.9530773043 | +2.24% |
| US-738 (ATCC | | |
| Accession No. | | |
| PTA-127449) | | |

TABLE 14

| | Yield | % Change Compared |
|---|---|---|
| Treatment | (Bushels/Acre) | to Untreated Control |
| Untreated Control | 207.672 | — |
| Acrocalymma vagum | 210.254 | +1.24% |
| US-738 (ATCC | | |
| Accession No. | | |
| PTA-127449) | | |

Acrocalymma vagum US-738 (ATCC Accession No. PTA-127449) increased both soil carbon and yield compared to the untreated control with corn.

### Example 11. Clonostachys rosea US-114 (ATCC Accession No. PTA-127443) Applied to Soybeans Increases Soil Carbon and Yield

### Background

To evaluate the effects of Clonostachys rosea US-114 (ATCC Accession No PTA-127443) on soil carbon and yield, two soybean field trials were conducted near Greenville. Mississippi, and Hutchinson, Kansas, in the United States.

### Materials and Methods

Clonostachys rosea US-114 (ATCC Accession No. PTA-127443) was applied directly to soybean seeds at a rate of about 1000 CFU per seed immediately prior to sowing with a neutral carrier. Untreated soybean seeds were sown as a control. Six replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the ratio of TOC at harvest (% wt/wt) to the TOC at baseline (o wt/wt). The percent change between treated and untreated groups was calculated by subtracting the mean carbon build of the untreated group from that of the treated group.

### Results

Results for TOC and yield are shown in Tables 15 and 16, respectively.

TABLE 15

| | | Mean | % Change |
|---|---|---|---|
| | | Carbon Build | Compared |
| | | (TOCₕₐᵣᵥₑₛₜ/ | to Untreated |
| Trial Site | Treatment | TOC_{baseline}) | Control |
| Greenville, | Untreated Control | 0.7030269815 | — |
| Mississippi | | | |
| Greenville, | Clonostachys rosea | 0.7168988606 | +1.39% |
| Mississippi | US-114 (ATCC | | |
| | Accession No. | | |
| | PTA-127443) | | |
| Hutchinson, | Untreated Control | 0.7195190693 | — |
| Kansas | | | |
| Hutchinson, | Clonostachys rosea | 0.7635417553 | +4.40% |
| Kansas | US-114 (ATCC | | |
| | Accession No. | | |
| | PTA-127443) | | |

TABLE 16

| | | | % Change |
|---|---|---|---|
| | | | Compared |
| | | Yield | to Untreated |
| Trial Site | Treatment | (Bushels/Acre) | Control |
| Greenville, | Untreated Control | 65.59833333 | — |
| Mississippi | | | |
| Greenville, | Clonostachys rosea | 69.01666667 | +5.21% |
| Mississippi | US-114 (ATCC | | |
| | Accession No. | | |
| | PTA-127443) | | |
| Hutchinson, | Untreated Control | 58 | — |
| Kansas | | | |
| Hutchinson, | Clonostachys rosea | 59.275 | +2.20% |
| Kansas | US-114 (ATCC | | |
| | Accession No. | | |
| | PTA-127443) | | |

Clonostachys rosea US-114 (ATCC Accession No. PTA-127443) increased both soil carbon and yield compared to the untreated control with soybeans.

### Example 12. Clonostachys rosea US-114 (ATCC Accession No. PTA-127443) Applied to Corn Increases Soil Carbon

### Background

To evaluate the effects of Clonostachys rosea US-114 (ATCC Accession No. PTA-127443) on soil carbon, a con field trial was conducted near Troy, Ohio, in the United States.

### Materials and Methods

Clonostachys rosea US-114 (ATCC Accession No. PTA-127443) was applied directly to corn seeds at a rate of about 1000 CFU per seed immediately prior to sowing with a neutral carrier Untreated corn seeds were sown as a control. Six replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the ratio of TOC at harvest (% wt/wt) to the TOC at baseline (% wt/wt). The percent change between treated and untreated groups was calculated by subtracting the mean carbon build of the untreated group from that of the treated group.

### Results

Results for TOC are shown in Table 17. The yield observed with the treated and untreated control plants was not statistically different.

TABLE 17

| | Mean Carbon Build | % Change Compared |
|---|---|---|
| Treatment | (TOCₕₐᵣᵥₑₛₜ/TOC_{baseline}) | to Untreated Control |
| Untreated Control | 1.094797396 | — |
| Clonostachys rosea | 1.114818522 | +2.00% |
| US-114 (ATCC | | |
| Accession No. | | |
| PTA-127443) | | |

Clonostachys rosea US-114 (ATCC Accession No. PTA-127443) increased soil carbon compared to the untreated control with corn.

### Example 13. Clonostachys rosea US-712 (ATCC Accession No. PTA-127446) Applied to Barley Increases Soil Carbon

### Background

To evaluate the effects of Clonostachys rosea US-712 (ATCC Accession No. PTA-127446) on soil carbon, a barley field trial was conducted near Velva, North Dakota, in the United States.

### Materials and Methods

Clonostachys rosea US-712 (ATCC Accession No. PTA-127446) was applied directly to barley seeds at a rate of about 5000 CFU per seed immediately prior to sowing with a neutral carrier. Untreated soybean seeds were sown as a control. Six replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the ratio of TOC at harvest (% wt/wt) to the TOC at baseline (% wt/wt). The percent change between treated and untreated groups was calculated by subtracting the mean carbon build of the untreated group from that of the treated group.

### Results

Results for TOC are shown in Table 19. The yield observed with the treated and untreated control plants was not statistically different.

TABLE 18

| | Mean Carbon Build | % Change Compared |
|---|---|---|
| Treatment | (TOCₕₐᵣᵥₑₛₜ/TOC_{baseline}) | to Untreated Control |
| Untreated Control | 0.8937535767 | — |
| Clonostachys rosea | 0.9430716042 | +4.93% |
| US-712 (ATCC | | |
| Accession No. | | |
| PTA-127446) | | |

Clonostachys rosea US-712 (ATCC Accession No, PTA-127446) increased soil carbon compared to the untreated control with barley.

### Example 14. Trichoderma hamatum US-724 (ATCC Accession No. PTA-127448) Applied to Soybeans Increases Soil Carbon and Yield

### Background

To evaluate the effects of Trichoderma hamatum US-724 (ATCC Accession No. PTA-127448) on soil carbon and yield, a soybean field trial was conducted near York. Nebraska, in the United States.

### Materials and Methods

Trichoderma hamatum US-724 (ATCC Accession No. PTA-127448) was applied directly to corn seeds at a rate of about 1000 CFU per seed immediately prior to sowing with a neutral carrier. Untreated corn seeds were sown as a control. Five replicates were evaluated for each group. The average total organic carbon (TOC) in each group was determined at harvest. TOC measurements were performed with a LECO® instrument using combustion of carbon.

Soil samples were collected at or near planting (i.e., “baseline samples”) and at or near harvest (i.e., “harvest samples”), Mean carbon build was calculated as the ratio of TOC at harvest (% wt/wt) to the TOC at baseline (% wt/wt). The percent change between treated and untreated groups was calculated by subtracting the mean carbon build of the untreated group from that of the treated group.

### Results

Results for TOC and yield are shown in Tables 19 and 20, respectively.

TABLE 19

| | Mean Carbon Build | % Change Compared |
|---|---|---|
| Treatment | (TOCₕₐᵣᵥₑₛₜ/TOC_{baseline}) | to Untreated Control |
| Untreated Control | 1.010419188 | — |
| Trichoderma hamatum | 1.079328391 | +6.89% |
| US-724 (ATCC | | |
| Accession No. | | |
| PTA-127448) | | |

TABLE 20

| | Yield | % Change Compared |
|---|---|---|
| Treatment | (Bushels/Acre) | to Untreated Control |
| Untreated Control | 51.56666667 | — |
| Trichoderma hamatum | 53 | +2.78% |
| US-724 (ATCC | | |
| Accession No. | | |
| PTA-127448) | | |

Trichoderma hamatum US-724 (ATCC Accession No. PTA-127448) increased both soil carbon and yield compared to the untreated control with soybeans.

### Example 15. Trichoderma hamatum US-724 (ATCC Accession No. PTA-127448) Applied to Corn Increases Soil Carbon and Yield

### Background

To evaluate the effects of Trichoderma hamatum US-724 (ATCC Accession No. PTA-127448) on soil carbon, a corn field trial was conducted near York, Nebraska, in the United States.

### Materials and Methods

Trichoderma hamatum US-724 (ATCC Accession No. PTA-127448) was applied directly to corn seeds at a rate of about 1000 CFU per seed immediately prior to sowing with a neutral carrier. Untreated corn seeds were sown as a control. Five replicates were evaluated for each group. The average total organic carbon (TOC) it) each group was determined at harvest. TOC measurements were performed with a LECO® instrument using combustion of carbon.

Soil samples were collected at or near planting (i.e., “baseline samples”) and at or near harvest (i.e., “harvest samples”). Mean carbon build was calculated as the ratio of TOC at harvest (% wt/wt) to the TOC at baseline (% wt/wt). The percent change between treated and untreated groups was calculated by subtracting the mean carbon build of the untreated group from that of the treated group.

### Results

Results for TOC are shown in Table 21, The yield observed with the treated and untreated control plants was not statistically different.

TABLE 21

| | Mean Carbon Build | % Change Compared |
|---|---|---|
| Treatment | (TOCₕₐᵣᵥₑₛₜ/TOC_{baseline}) | to Untreated Control |
| Untreated Control | 0.7525281299 | — |
| Trichoderma hamatum | 0.9430748221 | +19.05% |
| US-724 (ATCC | | |
| Accession No. | | |
| PTA-127448) | | |

Trichoderma hamatum US-724 (ATCC Accession No. PTA-127448) increased soil carbon compared to the untreated control with corn.

### Example 16. Periconia macrospinosa AU-7083 (NMI Accession No. V22/01976) Applied to Wheat Increases Soil Carbon and Yield

### Background

To evaluate the effects of Periconia macrospinosa AU-7083 (NMI Accession No. V22/019796) on soil carbon and yield, a wheat field trial was conducted near Horsham, Victoria, in Australia.

### Materials and Methods

Periconia macrospinosa AU-7083 (NMI Accession No. V22/019796) was applied directly to wheat seeds at a rate of about 67 CFU per seed immediately prior to sowing with a neutral carrier. Untreated wheat seeds were sown as a control. Six replicates were evaluated for each group for carbon measurements and yield measurements. The yield and total organic carbon (TOC) in each group were determined at harvest. TOC measurements were performed with a LECO® instrument using combustion of carbon.

### Results

Results for TOC and yield are shown in Tables 22 and 23, respectively.

TABLE 22

| | TOC at Harvest | % Change Compared |
|---|---|---|
| Treatment | (% wt/wt) | to Untreated Control |
| Untreated Control | 1.39 | — |
| Periconia macrospinosa | 1.42 | +2.16% |
| AU-7083 (NMI | | |
| Accession No. | | |
| V22/019796) | | |

TABLE 23

| | Yield | % Change Compared |
|---|---|---|
| Treatment | (Tonnes/Hectare) | to Untreated Control |
| Untreated Control | 5.44 | — |
| Periconia macrospinosa | 3.49 | +0.92% |
| AU-7083 (NMI | | |
| Accession No. | | |
| V22/019796) | | |

Periconia macrospinosa AU-7083 (NMI Accession No. V22/019796) increased both soil carbon and yield compared to the untreated control with wheat.

### Example 17. Periconia macrospinosa AU-7083 (NMI Accession No. V22/019796) Applied to Canola Increases Soil Carbon and Yield

### Background

To evaluate the effects of Periconia macrospinosa AU-7083 (NMI Accession No. V22/019796) on soil carbon and yield, a canola field trial was conducted near Beverley, Western Australia, in Australia.

### Materials and Methods

Periconia macrospinosa AU-7083 (NMI Accession No. V22/019796) was applied directly to canola seeds at a rate of about 68 CFU per seed immediately prior to sowing with a neutral carrier. Untreated canola seeds were sown as a control. Six replicates were evaluated for each group for carbon and yield measurements. The yield and total organic carbon (TOC) in each group were determined at harvest. TOC measurements were performed with a LECO® instrument using combustion of carbon.

### Results

Results for TOC and yield are shown in Tables 24 and 25, respectively.

TABLE 24

| | TOC at Harvest | % Change Compared |
|---|---|---|
| Treatment | (% wt/wt) | to Untreated Control |
| Untreated Control | 1.27 | — |
| Periconia macrospinosa | 1.32 | +3.94% |
| AU-7083 (NMI | | |
| Accession No. | | |
| V22/019796) | | |

TABLE 25

| | Yield | % Change Compared |
|---|---|---|
| Treatment | (Tonnes/Hectare) | to Untreated Control |
| Untreated Control | 1.32 | — |
| Periconia macrospinosa | 1.37 | +3.79% |
| AU-7083 (NMI | | |
| Accession No. | | |
| V22/019796) | | |

Periconia macrospinosa AU-7083 (NMI Accession No. V22/019796) increased both soil carbon and yield compared to the untreated control with canola.

### Example 11. Periconia macrospinosa AU-7083 (NMI Accession No. V22/019796) Applied to Radish Increases Soil Carbon and Yield

### Background

To evaluate the effects of Periconia macrospinosa AU-7083 (NMI Accession No. V22/019796) on soil carbon and crop growth (i.e., dry biomass), a radish field trial was conducted near Naracoorte, South Australia, in Australia.

### Materials and Methods

Periconia macrospinosa AU-7083 (NMI Accession No. V22/019796) was applied directly to radish seeds at a rate of about 130 CFU per seed immediately prior to sowing with a neutral carrier. Untreated radish seeds were sown as a control. Six replicates were evaluated for each group for carbon measurements and dry biomass measurements. The dry biomass and total organic carbon (TOC) in each group were determined at harvest TOC measurements were performed with a LECO® instrument using combustion of carbon.

### Results

Results for TOC and yield are shown in Tables 26 and 27, respectively.

TABLE 26

| | TOC at Harvest | % Change Compared |
|---|---|---|
| Treatment | (% wt/wt) | to Untreated Control |
| Untreated Control | 0.60 | — |
| Periconia macrospinosa | 0.68 | +13.33% |
| AU-7083 (NMI | | |
| Accession No. | | |
| V22/019796) | | |

TABLE 27

| | Dry Biomass | % Change Compared |
|---|---|---|
| Treatment | (Grams/Hectare) | to Untreated Control |
| Untreated Control | 1207.50 | — |
| Periconia macrospinosa | 1302.50 | +7.87% |
| AU-7083 (NMI | | |
| Accession No. | | |
| V22/019796) | | |

Periconia macrospinosa AU-7083 (NMI Accession No. V22/019796) increased both soil carbon and yield compared to the untreated control with radishes.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### INCORPORATION BY REFERENCE

All references, articles, publications, patents, patent publications, and patent applications cited herein within the above text and/or cited below are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### REFERENCES

Alexander, D. B., & Zuberer, D. A. (1991). Use of chrome azurol S reagents to evaluate siderophore production by rhizosphere bacteria. Biology and Fertility of soils, 12(1), 39-45.
Almeida, L. F. J., Souza, I. F., Hurtarte, L. C. C., Teixeira, P. P. C., inagaki, T. M., Silva, I. R., Mueller, C. W. 2021. Forest litter constraints on the pathways controlling soil organic matter formation. Soil Biology and Biochemistry, 163, 106447.
Buss, W., Sharma, R., Ferguson, C., Borevitz, J. (2021) Soil organic carbon fractionation and metagenomics pipeline to link carbon content and stability with microbial composition—first results investigating fungal endophytes. Genomics https://doi.org/10.1101/2021.12.19.473394
Chenu, C., Rumpel, C., Lehmann, J. (2015) Methods for studying soil organic matter: nature, dynamics, spatial accessibility, and interactions with minerals. Soil Microbiology, Ecology and Biochemistry. https://hal.archives-ouvertes.ft/hal-01598830
Doilom, M., Guo, J. W., Phookamsak, R., Mortimer, P. E., Karunarathna, S. C., Dong, W., . . . & Xu, J. C. (2020). Screening of phosphate-solubilizing fungi from air and soil in Yunnan, China: four novel species in Aspergillus, Gongronella, Penicillium, and Talaromyces. Frontiers in microbiology, 11, 585215.
Feng, W., Plante, A. F., Six, J. (2013) Improving estimates of maximal organic carbon stabilization by fine soil particles. Biogeochemistry, 112, 81-93.
Friedlingstein, P, Jones, M. W., O'Sullivan, M., Andrew, R. M., Dorothee, C., Bakker, C. E., Hauck, J, Le Quere. C., Peters. G. P., Peters. W., Pongratz, J., Sitch, S., Canadell. J. G., Ciais, P., Jackson, R. B., Alin, S. R., Anthoni, P., Bates, N. R., Becker, M., Bellouin, N., Bopp, L., Chau. T. T. T., Chevallier, F., Chini, L. P., Cronin, M., Currie, K. I., Decharme. B., Djeutchouang, L. M., Dou, X., Evans W, Feely, R. A., Feng, L, Gasser, T., Gilfillan, D., Gkritzalis, T., Grassi, G., Gregor, L., Gruber, N., Gurses, O., Harris, I., Houghton, R. A., Hurtt, G. C., Ilyina, T., Luijkx, I. C., Jain, A. K., Jones, S. D., Kato, E., Kennedy, D., Goldewijk, K. K., Knauer, J., Korsbakken, J. I., Kortzinger, A., Landschutzer, P., Lauvset, S. K., Lefevre, N., Lienert, S., Liu, J., Marland, G., McGuire, P. C., Melton, J. R., Munro, D. R., Nabel, J. E. M. S., Nakaoka, S. I., Niwa, Y., Ono. T., Pierrot, D., Poulter, B., Rehder, G., Resplandy, L., Robertson, E., Rodenbeck, C., Rosan, T. M., Schwinger, J., Schwingshacki, C., Sefdrian, R., Sutton, A. J., Sweeney, C., Tanhua, T., Tans, P. P., Tian, H., Tilbrook, B., Tubiello, F., van der Werf, G., Vuichard, N., Wada, C., Wanninkhof, R., Watson, A. J., Willis, D., Wiltshire. A. J., Yuan, W., Yue, C., Yue, X., Zaehle. S. Zheng, J. (2021) Global Carbon Budget 2021. Earth Syst Sci Data 14: 1917-2005. https://doi.org/10.5194/essd-14-1917-2022
Hemingway, J., Rothman, D. H., Grant, K. E., Rosengard, S. Z., Elington, T. T., Derry, L. A., Galy V. V. (2019). Mineral protection regulates long-term global preservation of natural organic carbon. Nature, 570, 228-231).
Hider, R. C., & Kong, X, (2010). Chemistry and biology of siderophores. Natural product reports, 27(5), 637-657,
IPCC (2021) Summary for policymakers. In: V Masson-Delmotte, P Zhai, A Pirani. SL Connors, C PHan, S Berger, N Caud, Y Chen, L Goldfarb, M I Gomis, M Huang, K Leitzell, E Lonnoy, J B R Matthews, T K Maycock, T Waterfield, O Yelekçi, R Yu, B Zhou (eds) Climate Change 2021: The Physical Science Basis Contribution of Working Group I to the Sixth Assessment Report of the Intergovernmental Panel on Climate Change. Cambridge University Press.
Kleber, M, Eusterhues, K., Keituweit, M., Mikutta, C., Mikutta, R., Nico, P. S (2015). Mineral-organic associations: Formation, properties, and relevance in soil environments. Advances in Agronomy. 130, 3-140.
Lal, R. (2018) Digging deeper” A holistic perspective of factors affecting soil carbon sequestration in agroecosystems. Global Change Biology, 24, 3285-3301.
Lavallee, J. M., Soong, J. L, Cotrufo, M. F. (2019) Conceptualizing soil organic matter into particulate and mineral-associated forms to address global change in the 21st century. Global Change Biology, 26, 261-273.

Lugato, E., Lavallee, J. M., Haddix, M. L., Panagos, P., Cotrufo M. F. (2021): Different climate sensitivity of particulate and mineral-associated soil organic matter. Nature Geoscience, 14, 295-300,
Mayer M., Krause, H-M., Fliessbach, A., Mader, P., Steffens, M. (2022) Fertilizer quality and labile soil organic matter fractions are vital for organic carbon sequestration in temperate arable soils within a long-term trial in Switzerland. Geoderma, 426, 116080.
Mugerwa, M. T. T., McGee, P. A. (2017) Potential effect of melanised endophytic fungi on levels of organic carbon within an Alfisol. Soil Research 55, 245-252.
Poeplau, C., Don, A., Six, J., Kaiser, M., Benbi, D., Chenu, C., Cotrufo, M. F., Derrien, D., Gioacchini, P., Grand, S., Gregorich, E., Griepentrog, M., Gunina, A., Haddix, M., Kuzyakov, Y., Kuhnel, A., Macdonald, L. M., Soong, J., Trigalet, S., Vermeire, M-L., Rovira, P., van Wesemael, B., Wiesmeier, M., Yeasmin, S., Yevdokimov, L., Nieder, R. (2018) Isolating organic carbon fractions with varying turnover rates in temperate agricultural soils-A comprehensive method comparison. Soil Biology and Biochemistry, 125, 10-26.
Rocci, K. S., Lavallee, J. M., Stewart C. E., Cotrufo, M. F. (2021) Soil organic carbon response to global environmental change depends on its distribution between mineral-associated and particulate organic matter. Science of the Total Environment, 793, 148569.
Rodrigues, L. A. T., Giacomini, S. J., Diecknow, J., Cherubin, R., Ottonelli, A. S., Bayer, C. (2022) Carbon saturation deficit and litter quality drive the stabilization of litter-derived C in mineral-associated organic matter in long-term no-till soil. Catena, 219, 106590.
Sanderman, J., Hengt, T., Fiske, G., (2017) Soil carbon debt of 12,000 years of human land use. Proceedings of the National Academy of Sciences, 114, 9575-9580.
Schweizer, S. A., Mueller, C. W., Hoschen, C., Ivanov, P., Kogel-Knabner, I. (2021) The role of clay content and mineral surface area for soil organic carbon storage in an arable toposequence. Biogeochemistry, 156, 401-420.
Sharma, S. B., Sayyed, R. Z., Trivedi, M. H., & Gobi, T. A. (2013). Phosphate solubilizing microbes: sustainable approach for managing phosphorus deficiency in agricultural soils. SpringerPlus, 2(1), 1-14.
Six, J (1999). Recycling of sodium polytungstate used in soil organic matter studies. Soil Biology and Biochemistry, 31 (8), 1193-1196.

What is claimed is:

1. A method of increasing organic carbon in a soil, comprising:
inoculating the soil and/or a plant growing in the soil with one or more fungal strains,
wherein the one or more fungal strains has a nuclear ribosomal internal transcribed spacer 2 (ITS2) sequence that is at least 90% identical to the nucleotide sequence of any one of SEQ ID NOs: 1-11; and
the one or more fungal strains are in an effective amount to increase organic carbon in the soil compared to a non-inoculated control soil.

2. The method of claim 1, wherein the one or more fungal strains are selected from the group consisting of: Periconia macrospinosa AU-7083 (NMI Accession No. V22/019796), Acrocalymma vagum US-738 (ATCC Accession No. PTA-127449), Acrocalymma vagum US-445 (ATCC Accession No. PTA-127444), Leptodontidium orchidola US-210 (ATCC Accession No. PTA-127441), Leptodontidium orchidola US-70 (ATCC Accession No. PTA-127439), Leptodontidium orchidola US-1188 (ATCC Accession No. PTA-127640), Leptodontidium orchidola US-1202 (ATCC Accession No. PTA-127639), Clonostachys rosea US-114 (ATCC Accession No. PTA-127443), Clonostachys rosea US-712 (ATCC Accession No PTA-127446), Trichoderma hamatum US-724 (ATCC Accession No. PTA-127448), Trichoderma longipile/spirale US-77 (ATCC Accession No. PTA-127440), and a mutant thereof having all identifying characteristics of the respective strain.

3. The method of claim 1 or 2, further comprising an initial step of identifying the soil as having a soil organic carbon (SOC) level below a threshold.

4. The method of claim 3, wherein the threshold is a SOC level (% wt/wt) below 5%, 4%, 3%, 2%, 1%, 0.90, 0.8%, 0.7%, 0.6%, 0.5%, 0.4%, 0.3%, 0.2%, or 0.1%.

5. The method of any one of claims 1 to 4, wherein the soil and/or plant are non-native to the one or more fungal strains.

6. The method of claim 5, wherein the non-native plant is selected from the group consisting of wheat, rice, corn (maize), canola, rye, oats, barley, sorghum, millet, flax, hemp, jute, cotton, soybeans, alfalfa, clover, peanuts, lentils, lupins, peas, and chickpea.

7. The method of claim 5, wherein the non-native plant is selected from the group consisting of Lucerne, arrow leaf clover, Balansa clover, chicory, plantain, Phalaris, cocksfoot, fescue, prairie grass, Warrego summer grass, Italian rye grass, perennial rye grass, Biserrula, Serradella, Gland clover, Bladder clover, switchgrass, radish, medic, buckwheat, cow pea, lablab, sunn hemp, sunflower, tillage radish, and subterranean clover.

8. The method of any one of claims 1 to 7, wherein the organic carbon in the soil is increased in the stable forms of aggregate carbon fraction (AggC), aggregate occluded particulate organic C (oPOC), and/or mineral-associated organic carbon (MAOC).

9. A method for sequestering atmospheric carbon for storage as organic carbon in a soil, comprising:
inoculating the soil and/or a plant growing in the soil with one or more fungal strains,
wherein the one or more fungal strains has a nuclear ribosomal internal transcribed spacer 2 (ITS2) sequence that is at least 90% identical to the nucleotide sequence of any one of SEQ ID NOs: 1-1; and
the one or more fungal strains are in an effective amount to increase sequestered atmospheric carbon in the soil compared to a non-inoculated control soil.

10. The method of claim 9, wherein the one or more fungal strains are selected from the group consisting of: Periconia macrospinosa AU-7083 (NMI Accession No. V22/019796), Acrocalymma vagum US-738 (ATCC Accession No. PTA-127449), Acrocalymma vagum US-445 (ATCC Accession No. PTA-127444), Leptodontidium orchidola US-210 (ATCC Accession No. PTA-127441), Leptodontidium orchidola US-70 (ATCC Accession No. PTA-127439), Leptodontidium orchidola US-1188 (ATCC Accession No. PTA-127640), Leptodontidium orchidola US-1202 (ATCC Accession No. PTA-127639), Clonostachys rosea US-114 (ATCC Accession No. PTA-127443), Clonostachys rosea US-712 (ATCC Accession No PTA-127446), Trichoderma hamatum US-724 (ATCC Accession No. PTA-127448), Trichoderma longipile/spirale US-77 (ATCC Accession No. PTA-127440), and a mutant thereof having all identifying characteristics of the respective strain.

11. The method of claim 9 or 10, wherein the plant is non-native to the one or more fungal strains.

12. The method of claim 11, wherein the non-native plant is selected from the group consisting of wheat, rice, corn (maize), canola, rye, oats, barley, sorghum, millet, flax, hemp, jute, cotton, soybeans, alfalfa, clover, peanuts, lentils, lupins, peas, and chickpea.

13. The method of claim 11, wherein the non-native plant is selected from the group consisting of Lucerne, arrow leaf clover, Balansa clover, chicory, plantain, Phalaris, cocksfoot, fescue, prairie grass, Warrego summer grass, Italian rye grass, perennial rye grass, Biserrula, Serradella, Gland clover, Bladder clover, switchgrass, radish, medic, buckwheat, cow pea, lablab, sunn hemp, sunflower, tillage radish, and subterranean clover.

14. The method of any one of claims 9 to 13, wherein the organic carbon in the soil is increased in the stable forms of aggregate carbon fraction (AggC), aggregate occluded particulate organic C (oPOC), and/or mineral-associated organic carbon (MAOC).

15. A method of enhancing plant growth, comprising:
applying to a plant, a plant part, or the locus surrounding the plant, one or more fungal strains,
wherein the one or more fungal strains has a nuclear ribosomal internal transcribed spacer 2 (ITS2) sequence that is at least 90% identical to the nucleotide sequence of any one of SEQ ID NOs: 1-11; and
the one or more fungal strains are in an effective amount to enhance the growth of the plant as compared to an untreated control plant.

16. The method of claim 15, wherein plant growth is enhanced by increased phosphate solubilization or siderophore production by the one or more fungal strains.

17. The method of claim 15 or 16, wherein the one or more fungal strains are selected from the group consisting of: Periconia macrospinosa AU-7083 (NMI Accession No. V22/019796), Acrocalymma vagum US-738 (ATCC Accession No. PTA-127449), Acrocalymma vagum US-445 (ATCC Accession No. PTA-127444), Leptodontidium orchidola US-210 (ATCC Accession No. PTA-127441), Leptodontidium orchidola US-70 (ATCC Accession No. PTA-127439), Leptodontidium orchidola US-1188 (ATCC Accession No. PTA-127640), Leptodontidium orchidola US-1202 (ATCC Accession No. PTA-127639), Clonostachys rosea US-114 (ATCC Accession No. PTA-127443), Clonostachys rosea US-712 (ATCC Accession No PTA-127446), Trichoderma hamatum US-724 (ATCC Accession No. PTA-127448), Trichoderma longipile/spirale US-77 (ATCC Accession No. PTA-127440), and a mutant thereof having all identifying characteristics of the respective strain.

18. The method of any one of claims 15 to 17, wherein the plant exhibits at least one of increased root number, increased root length, increased root mass, increased root volume, increased leaf area, increased leaf number, increased pod number, increased plant height, increased shoot mass, increased chlorophyll content, increased nodulation, and increased yield, as compared to an untreated control plant.

19. The method of any one of claims 15 to 18, wherein the plant is non-native to the one or more fungal strains.

20. The method of claim 19, wherein the non-native plant is selected from the group consisting of wheat, rice, corn (maize), canola, rye, oats, barley, sorghum, millet, flax, hemp, jute, cotton, soybeans, alfalfa, clover, peanuts, lentils, lupins, peas, and chickpea.

21. The method of claim 19, wherein the non-native plant is selected from the group consisting of Lucerne, arrow leaf clover, Balansa clover, chicory, plantain, Phalaris, cocksfoot, fescue, prairie grass, Warrego summer grass, Italian rye grass, perennial rye grass, Biserrula, Serradella, Gland clover, Bladder clover, switchgrass, radish, medic, buckwheat, cow pea, lablab, sunn hemp, sunflower, tillage radish, and subterranean clover.

22. A plant, plant part or plant seed associated with a composition comprising:
one or more fungal strains having a nuclear ribosomal internal transcribed spacer 2 (ITS2) sequence that is at least 90% identical to the nucleotide sequence of any one of SEQ 1D NOs: 1-11; and an agriculturally acceptable carrier;
wherein the composition is applied or coated on at least a portion of an outer surface of the plant, plant part or plant seed.

23. The plant, plant part or plant seed of claim 22, wherein the one or more fungal strains are selected from the group consisting of: Periconia macrospinosa AU-7083 (NMI Accession No. V22/019796), Acrocalymma vagum US-738 (ATCC Accession No. PTA-127449), Acrocalymma vagum US-445 (ATCC Accession No. PTA-127444), Leptodontidium orchidola US-210 (ATCC Accession No. PTA-127441), Leptodontidium orchidola US-70 (ATCC Accession No. PTA-127439), Leptodontidium orchidola US-1188 (ATCC Accession No. PTA-127640), Leptodontidium orchidola US-1202 (ATCC Accession No. PTA-127639), Clonostachys rosea US-114 (ATCC Accession No. PTA-127443), Clonostachys rosea US-712 (ATCC Accession No PTA-127446), Trichoderma hamatum US-724 (ATCC Accession No. PTA-127448), Trichoderma longipile/spirale US-77 (ATCC Accession No. PTA-127440), and a mutant thereof having all identifying characteristics of the respective strain.

24. The plant, plant part or plant seed of claims 22 or 23, wherein the plant, plant part or plant seed is non-native to the one or more fungal strains.

25. The plant, plant part or plant seed of claim 24, wherein the non-native plant, plant part or plant seed is selected from the group consisting of wheat, rice, corn (maize), canola, rye, oats, barley, sorghum, millet, flax, hemp, jute, cotton, soybeans, alfalfa, clover, peanuts, lentils, lupins, peas, and chickpea.

26. The plant, plant part or plant seed of claim 24, wherein the non-native plant, plant part or plant seed is a pasture crop or cover crop selected from the group consisting of Lucerne, arrow leaf clover, Balansa clover, chicory, plantain, Phalaris, cocksfoot, fescue, prairie grass, Warrego summer grass, Italian rye grass, perennial rye grass, Biserrula, Serradella, Gland clover, Bladder clover, switchgrass, radish, medic, buckwheat, cow pea, lablab, sunn hemp, sunflower, tillage radish, and subterranean clover.

27. The plant, plant part or plant seed of any one of claims 22 to 26, wherein the composition is formulated as a powder, a pellet, granules, emulsion, colloid, suspension or solution.

28. The plant, plant part or plant seed of any one of claims 22 to 27, wherein the one or more fungal strains are present in the composition at a concentration of at least 10³ CFU per millilitre or gram.

29. A bag or container comprising the plant seed of any one of claims 22 to 28.

30. A kit comprising the plant seed according to of any one of claims 22 to 28.

31. A cell or a biologically pure culture of one or more fungal strains selected from the group consisting of Periconia macrospinosa AU-7083 (NMI Accession No. V22/019796), Acrocalymma vagum US-738 (ATCC Accession No. PTA-127449), Acrocalymma vagum US-445 (ATCC Accession No. PTA-127444), Leptodontidium orchidola US-210 (ATCC Accession No. PTA-127441), Leptodontidium orchidola US-70 (ATCC Accession No. PTA-127439), Leptodontidium orchidola US-1188 (ATCC Accession No. PTA-127640), Leptodontidium orchidola US-1202 (ATCC Accession No. PTA-127639), Clonostachys rosea US-114 (ATCC Accession No. PTA-127443), Clonostachys rosea US-712 (ATCC Accession No PTA-127446), Trichoderma hamatum US-724 (ATCC Accession No. PTA-127448), Trichoderma longipile/spirale US-77 (ATCC Accession No. PTA-127440), and a mutant thereof having all identifying characteristics of the respective strain.

32. An agricultural composition comprising the cell or a biologically pure culture of claim 31 and, optionally, an agriculturally acceptable carrier.

33. The agricultural composition of claim 32, wherein the agricultural composition is applied or coated on at least a portion of an outer surface of a plant, plant part or plant seed.

34. A bioorganic soil conditioner comprising the cell or a biologically pure culture of claim 31 and, optionally, an agriculturally acceptable carrier.


SEQUENCE LISTING (11 sequences)

SEQ ID NO: 1 - DNA, 347 bp, synthetic construct
  source 1..347: mol_type = other DNA
gcatcgatgaagaacgcagcgaaatgcgataagtagtgtgaattgcagaattcagtgaatcatcgaatctttgaacgcacattgcggccataggtattcctttggccatgcctgttcgagcgtcatttacaccctcaagcctagcttggtgttgggcgtctgtcccgccgttttcgcgcgcggactcgcctcaaagtcattggcggcggtcgtgccggccccctcgcgcagcacatttgcgcttctcggaggcccggcggatccgcgctccagcaagacctttcacgacttgacctcggatcaggtagggatacccgctgaacttaagcatatcaataagcggagga

SEQ ID NO: 2 - DNA, 750 bp, synthetic construct
  source 1..750: mol_type = other DNA
tgcgggaacaccctaaagacctcaacaccaagcgtcatgggaaaccatggcgtggccgagctaatagccctgggtatggtaacagcttgaggtatgaagccttcgcaaggaggccgaaatgggcaatccgcagccaagtcctaacgtgctcgaaaccgagtgccatggatgctgttcacaggccaaatggtagtgggtgactcttgcgagttgcttaagatatggtcgggccccttcagaaatgtgggggataagcttacgcttctccaaaccgttccgtaggtgaacctgcggaaggatcattaacgatttcggtgtaaaaaaccgttttctacctatgtctacgcgtaccacatgtttcctcggggggcttgccccccgctaggaccctttatcaaacctttttgtaatagcagtcagcgtctgatactaagttaattattaaaactttcaacaatggatctcttggttctggcatcgatgaagaacgcagcgaaatgcgataagtagtgtgaattgcagaattcagtgaatcatcgaatctttgaacgcacattgcgccccttggtattccatggggcatgcctgttcgagcgtcatttgaaccctcaagctctgcttggtgttgggtgtttgtcccgccattgcgcgtgactcgccttaagcaattggcagccatgtaatcccgcctttgagcgcagcacattgcgtactctctactggacatgggcatccagaagccttattttt

SEQ ID NO: 3 - DNA, 340 bp, synthetic construct
  source 1..340: mol_type = other DNA
tcctccgcttattgatatgcttaagttcagcgggtatccctacctgatccgaggtcaagagtaaaaaataaggcttctggatgcccatgtcccagtagagagtacgcaatgtgctgcgctcaaagccggattacatggctgccaattgctttaaggcgagtccacgcgcaatggcgggacaaacacccaacaccaagcagagcttgagggttcaaatgacgctcgaacaggcatgccccatggaataccaaggggcgcaatgtgcgttcaaagattcgatgattcactgaattctgcaattcacactacttatcgcatttcgctgcgttcttcatcgatg

SEQ ID NO: 4 - DNA, 334 bp, synthetic construct
  source 1..334: mol_type = other DNA
tcctccgcttattgatatgcttaagttcagcgggtatccctacctgatccgaggtcaaccttaaaaaaattaggggttgctggcaagtagacctaccggactcaatcgcgaggagtattactacgcgtagagccgacaggcaccgccactgattttaggggccgcgaaaccgcgaaccccaataccaagcgagagcttgagtggttataatgacgctcgaacaggcatgccccccggaataccagagggcgcaatgtgcgttcaaagattcgatgattcactgaattctgcaattcacattacttatcgcatttcgctgcgttcttcatcgatg

SEQ ID NO: 5 - DNA, 336 bp, synthetic construct
  source 1..336: mol_type = other DNA
gcatcgatgaagaacgcagcgaaatgcgataagtaatgtgaattgcagaattcagtgaatcatcgaatctttgaacgcacattgcgccctctggtattccggggggcatgcctgttcgagcgtcattataaccactcaagctctcgcttggtattggggttcgcggtttcgcggcccctaaaatcagtggcggtgcctgtcggctctacgcgtagtaatactcctcgcgattgagtccggtaggtctacttgccagcaacccctaatttttttttaaggttgacctcggatcaggtagggatacccgctgaacttaagcatatcaataagcggagg

SEQ ID NO: 6 - DNA, 601 bp, synthetic construct
  source 1..601: mol_type = other DNA
ctgcggaggatcattactagagcaaaggatagacagcgcccgcggagctcgctcccggggccaccctactccggtagggtttagagtcgtcgggcctctcggagaagctcggtcctgaactccacccttgaataaactacctttgttgctttggcgggccgcctcgtgccagcggcttcggctgttgagtgcccgccagaggaccacaactcttgtttttagtgatgtctgagtactatataatagttaaaactttcaacaacggatctcttggttctggcatcgatgaagaacgcagcgaaatgcgataagtaatgtgaattgcagaattcagtgaatcatcgaatctttgaacgcacattgcgccctctggtattccggggggcatgcctgttcgagcgtcattataaccactcaagctctcgcttggtattggggttcgcggtttcgcggctcctaaaatcagtggcggtgcctatcggctctacgcgtagtaatactcctcgcgattgagtccggtaggtctacttgccagcaacccctaattttttaaggttgacctcggatcaggtagggatacccgctgaacttaagcatatca

SEQ ID NO: 7 - DNA, 664 bp, synthetic construct
  source 1..664: mol_type = other DNA
cttggtcatttagaggaagtaaaagtcgtaacaaggtttccgtaggtgaacctgcggaaggatcattactagagcaaaggatagacagcgcccgcggagctcgctcccggggctaccctactccggtagggtttagagtcgtcgggcctctcagagaagctcggtcctgaactccacccttgaataaactacctttgttgctttggcgggccgcctcgtgccagcggctttggctgttgagtgcccgccagaggaccacaactcttgtttttagtgatgtctgagtactatataatagttaaaactttcaacaacggatctcttggttctggcatcgatgaagaacgcagcgaaatgcgataagtaatgtgaattgcagaattcagtgaatcatcgaatctttgaacgcacattgcgccctctggtattccggggggcatgcctgttcgagcgtcattataaccactcaagctctcgcttggtattggggttcgcggtttcgcggcccctaaattcagtggcggtgcctatcggctctacgcgtagtaatactcctcgcgattgagtccggtaggtctacttgccagtaacccctaattttttaaggttgacctcggatcaggtagggatacccgctgaacttaagcatatcaataagcggagg

SEQ ID NO: 8 - DNA, 352 bp, synthetic construct
  source 1..352: mol_type = other DNA
gcatcgatgaagaacgcagcgaaatgcgataagtaatgtgaattgcagaattcagtgaatcatcgaatctttgaacgcacattgcgcccgccagtattctggcgggcatgcctgtctgagcgtcatttcaaccctcatgcccctagggcgtggtgttggggatcggccaaagcccgcgagggacggccggcccctaaatctagtggcggacccgtcgtggcctcctctgcgaagtagtgatattccgcatcggagagcgacgagcccctgccgttaaacccccaactttccaaggttgacctcagatcaggtaggaatacccgctgaacttaagcatatcaataagcggagg

SEQ ID NO: 9 - DNA, 543 bp, synthetic construct
  source 1..543: mol_type = other DNA
gcggagggatcattaccgagtttacaactcccaaacccatgtgaacatacctactgttgcttcggcgggattgccccgggcgcctcgtgtgccccggatcaggcgcccgcctaggaaacttaattcttgttttattttggaatcttctgagtagtttttacaaataaataaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtctgagcgtcatttcaaccctcatgcccctagggcgtggtgttggggatcggccaaagcccgcgagggacggccggcccctaaatctagtggcggacccgtcgtggcctcctctgcgaagtagtgatattccgcatcggagagcgacgagcccctgccgttaaacccccaactttccaaggttgacctcagatcaggtaggaatacccgctgaacttaagcatatcaa

SEQ ID NO: 10 - DNA, 584 bp, synthetic construct
  source 1..584: mol_type = other DNA
cggagggatcattaccgagtttacaactcccaaacccaatgtgaacgttaccaaactgttgcctcggcggggtcacgccccgggtgcgtaaaagccccggaaccaggcgcccgccggaggaaccaaccaaactctttctgtagtcccctcgcggacgtatttcttacagctctgagcaaaaattcaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccgggggatcggcgttggggatcgggacccctcaccgggtgccggccctgaaatacagtggcggtctcgccgcagcctctcctgcgcagtagtttgcacaactcgcaccgggagcgcggcgcgtccacgtccgtaaaacacccaacttctgaaatgttgacctcggatcaggtaggaatacccgctgaacttaagcatatcaataagcggagga

SEQ ID NO: 11 - DNA, 353 bp, synthetic construct
  source 1..353: mol_type = other DNA
gcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccggggggtcggcgttggggatcggccctttacggggccggccccgaaatacagtggcggtctcgccgcagcctctcctgcgcagtagtttgcacactcgcatcgggagcgcggcgcgtccattgccgttaaacacccaactttctgaaatgttgacctcggatcaggtaggaatacccgctgaacttaagcatatcaataagcggagg